(19) United States
(12) Patent Application Publication
(10) Pub. No.: US 2026/0080799 A1
(43) Pub. Date: Mar. 19, 2026

(54) SYSTEM AND METHOD FOR PROVIDING IMMERSIVE MEDICAL TRAINING WITH A VIRTUAL PATIENT

(71) Applicant: Anatomage Inc., Santa Clara, CA (US)

(72) Inventor: Woncheol Choi, San Jose, CA (US)

(21) Appl. No.: 19/329,106

(22) Filed: Sep. 15, 2025

Related U.S. Application Data
(60) Provisional application No. 63/695,336, filed on Sep. 16, 2024.
(60) Provisional application No. 63/831,622, filed on Jun. 27, 2025.
(60) Provisional application No. 63/831,992, filed on Jun. 27, 2025.

(51) Int. Cl. G09B 9/00; G16H 10/60

(52) U.S. Cl. CPC G09B 9/00; G16H 10/60

(57) ABSTRACT

An electronic system and method for providing interactive educational content and immersive learning experience are disclosed. According to one embodiment,?

### CROSS-REFERENCE TO RELATED APPLICATIONS

This application claims priority to U.S. Provisional Patent Application No. 63/695,336 filed Sep. 16, 2024, U.S. Provisional Patent Application No. 63/831,622 filed Jun. 27, 2025, and U.S. Provisional Patent Application No. 63/831,992 filed Jun. 27, 2025, the disclosures of which are incorporated by reference in their entireties.

### FIELD

The field of the present disclosure relates generally to a system and method for providing content, and more particularly, to a system and method for providing interactive educational content and immersive learning and training experience.

### BACKGROUND

Science, Technology, Engineering, and Mathematics (STEM) education is essential to educate disciplines and prepare students for the complexities of the modern world. However, integrating STEM education into conventional classrooms presents several significant challenges. These difficulties stem from various factors such as outdated curricula, aging facilities, equipment, and resources, a lack of educators and specialties.

One of the major challenges for STEM education is the outdated curricula in the era of digital worlds. Conventional educational systems often follow curricula that may not adequately address the dynamic and interactive nature of the STEM subjects and educational trends. These curricula may be too old and slow to adapt to emerging technologies that are readily available outside of the classroom. As a result, students may receive outdated education or even be forfeited opportunity to learn using the latest educational tools and technology that would greatly help developing relevant skills and knowledge.

Additionally, conventional classrooms suffer from a lack of proper resources.

Effective STEM education frequently requires specialized equipment, software, and materials that are not always available in typical school settings. For example, hands-on experiments, robotics kits, and advanced computing technology can be prohibitively expensive. Schools with limited budgets may struggle to provide these necessary resources, thereby hindering students from deeply engaging with the STEM subjects.

Another significant issue is the scarcity of professional training and development opportunities for educators. Teachers often enter the profession with limited training in STEM disciplines, and opportunities for their professional development in these areas are quite limited. Without adequate training, teachers may feel unprepared to deliver complex STEM content or incorporate innovative teaching strategies to their classroom. In addition, the teachers' workload to prepare and maintain and update the traditional STEM curricula can be an intimidating task and oftentimes beyond their ability. This can lead to less effective learning and training experiences for students because educators may not be properly trained or equipped with resources and tools to inspire their students in exploring STEM subjects.

### SUMMARY

An electronic system and method for providing interactive educational content and immersive learning and training experience are disclosed.

According to one embodiment, an immersive system includes: a computing system including a microphone and a speaker; and a surround screen. The computing system provides interactive scenes of a virtual hospital including a virtual patient on the surround screen. The virtual hospital includes one or more of hospital facilities, and a user is adapted to move around the one or more of hospital facilities. The virtual patient has a symptom or has a medical condition, and the user is further adapted to interact with the virtual patient via a voice command input through the microphone, an auditory output from the speaker, and the interactive scenes displayed by the surround screen.

The virtual patient may be an Artificial Intelligence (AI)-generated patient based on clinical patient data.

The surround screen may display a medical chart or a medical image of the virtual patient at a request of the user.

The immersive system may further include a medical tool that the user can touch and manipulate.

A digital counterpart of the medical tool may be updated on the interactive scenes displayed on the surround screen based on a position and an orientation of the medical tool.

The immersive system may further include a plurality of input devices to receive an input from the user, wherein the plurality of input devices includes one or more of a remote control, a joystick, a ring, a virtual reality (VR) device, a keyboard, a camera, a touchscreen, a trackpad, mouse, a game controller, and a foot pedal.

Based on consultation with the virtual patient, the user may treat the virtual patient with a medication, order a test, or a medical procedure.

The user's treatment on the virtual patient may be graded in a medical training session.

The one or more of hospital facilities may include a reception area, an administration department, a billing department, a conference room, a training room, cafeteria, an emergency room, a patient ward, an intensive care unit, an operating room, pharmacy, or an imaging room.

The surround screen may include a first display positioned on a left side, a second display positioned on a right side, and a third display positioned between the first display and the second display, and the first display, the second display, and the third display provide 270-degree immersive experience to the user.

According to one embodiment, a method of providing an immersive experience to a user includes: providing a computing system that includes a microphone and a speaker and a surround screen; providing interactive scenes of a virtual hospital including a virtual patient on the surround screen, wherein the virtual patient has a symptom or has a medical condition; and updating the scenes of the virtual hospital based on interaction between the user and the virtual patient via a voice command input through the microphone, an auditory output from the speaker, and the interactive scenes displayed by the surround screen. The virtual hospital includes one or more of hospital facilities, and a user is adapted to move around the one or more of hospital facilities.

The virtual patient may be an Artificial Intelligence (AI)-generated patient based on clinical patient data.

The method may further include displaying a medical chart or a medical image of the virtual patient on the surround screen at a request of the user.

The method may further include receiving an input from the user using a medical tool that can be touched and manipulated by the user.

The method may further include displaying and updating a digital counterpart of the medical tool on the interactive scenes on the surround screen based on a position and an orientation of the medical tool.

The method may further include receiving an input from the user using a plurality of input devices, wherein the plurality of input devices includes one or more of a remote control, a joystick, a ring, a virtual reality (VR) device, a keyboard, a camera, a touchscreen, a trackpad, mouse, a game controller, and a foot pedal.

The method may further include receiving an input from the user after consulting with the virtual patient, the input relates to treating the virtual patient with a medication, order a test, or a medical procedure.

The method may further include grading the user's treatment on the virtual patient in a medical training session.

The one or more of hospital facilities may include a reception area, an administration department, a billing department, a conference room, a training room, cafeteria, an emergency room, a patient ward, an intensive care unit, an operating room, pharmacy, or an imaging room.

The surround screen may include a first display positioned on a left side, a second display positioned on a right side, and a third display positioned between the first display and the second display, and the first display, the second display, and the third display provide 270-degree immersive experience to the user.

The above and other features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The accompanying drawings, which are included as part of the present specification, illustrate various embodiment of the present disclosure and together with the general description given above and the detailed description of the embodiment given below serve to explain and teach the principles of the present disclosure.

FIG. 1 illustrates an example of a classroom in which educational content is provided by a digital table, according to one embodiment;

FIG. 2 shows an example of a digital table, according to one embodiment;

FIG. 3 shows examples of user interface controls usable in a digital experiment, according to one embodiment;

FIG. 4 shows examples of buttons usable in a digital experiment, according to one embodiment;

FIG. 5 shows an example scene of the Fruit Fly Genetics experiment, according to one embodiment;

FIG. 6 shows an example procedure of the Fruit Fly Genetics experiment, according to one embodiment;

FIG. 7 shows an example scene of the Mass Spectrometer experiment, according to one embodiment;

FIG. 8 shows an example procedure of the Mass Spectrometer experiment, according to one embodiment;

FIG. 9 shows an example scene of the Electric Circuit experiment, according to one embodiment;

FIG. 10 shows an example procedure of the Electric Circuit experiment, according to one embodiment;

FIG. 11 shows an example scene of the Black Hole experiment, according to one embodiment;

FIG. 12 shows an example procedure of the Black Hole experiment, according to one embodiment;

FIG. 13 shows an immersive system for providing interactive content, according to another embodiment;

FIG. 14 is a block diagram of an exemplary computing system suitable for implementing an embodiment of the present system; and

FIG. 15 illustrates an example of a virtual hospital room, according to one embodiment.

It should be noted that the figures are not necessarily drawn to scale and that elements of structures or functions are generally represented by reference numerals for illustrative purposes throughout the figures. It also should be noted that the figures are only intended to facilitate the description of the various embodiments described herein. The figures do not describe every aspect of the teachings described herein and do not limit the scope of the claims.

### DETAILED DESCRIPTION

An electronic system and method for providing interactive educational content and immersive learning and training experience are disclosed.

In the following description, for purposes of clarity and conciseness of the description, not all of the numerous components shown in the schematic are described. The numerous components are shown in the drawings to provide a person of ordinary skill in the art a thorough enabling disclosure of the present disclosure. The operation of many of the components would be understood to one skilled in the art, and it will be apparent to one skilled in the art that these specific details are not required to practice the teachings of the present disclosure.

Some portions of the detailed descriptions herein are presented in terms of algorithms and symbolic representations of operations on data bits within a computer memory. These algorithmic descriptions and representations are used by those skilled in the data processing arts to effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the below discussion, it is appreciated that throughout the description, discussions utilizing terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The algorithms presented herein are not inherently related to any particular computer or other apparatus. Various general-purpose systems, computer servers, or personal computers may be used with programs in accordance with the teachings herein, or it may prove convenient to construct a more specialized apparatus to perform the required method steps. The required structure for a variety of these systems will appear from the description below. It will be appreciated that a variety of programming languages may be used to implement the teachings of the disclosure as described herein.

Each of the additional features and teachings disclosed herein can be utilized separately or in conjunction with other features and teachings to provide the present system and method. Representative examples utilizing many of these additional features and teachings, both separately and in combination, are described in further detail with reference to the attached drawings. This detailed description is merely intended to teach a person of skill in the art further details for practicing various aspects of the present teachings and is not intended to limit the scope of the claims. Therefore, combinations of features disclosed in the following detailed description may not be necessary to practice the teachings in the broadest sense and are instead taught merely to describe particularly representative examples of the present teachings.

Moreover, the various features of the representative examples and the dependent claims may be combined in ways that are not specifically and explicitly enumerated to provide additional useful embodiments of the present teachings. In addition, it is expressly noted that all features disclosed in the description and/or the claims are intended to be disclosed separately and independently from each other for the purpose of original disclosure, as well as for the purpose of restricting the claimed subject matter independent of the compositions of the features in the embodiments and/or the claims. It is also expressly noted that all value ranges or indications of groups of entities disclose every possible intermediate value or intermediate entity for the purpose of original disclosure, as well as for the purpose of restricting the claimed subject matter. It is also expressly noted that the dimensions and the shapes of the components shown in the figures are designed to help understand how the present teachings are practiced but are not intended to limit the dimensions and the shapes shown in the examples.

The present system and method provide interactive educational contents and immersive learning experience. According to one embodiment, the system and method provide a digital laboratory platform using a large touchscreen interface. The system and method enable students to perform a wide range of science experiments in an interactive and immersive manner to help them to fully and deeply engage with the provided educational contents.

According to one embodiment, the present system is provided in a form of a digital table. The digital table may include a large display for displaying educational contents. The digital table may be portable using a mechanism (e.g., wheels) for moving in or around a classroom. The digital table provides students with a hyper-realistic and tactile experience, simulating real-world experimentation digitally in a safe and clean environment that is otherwise impossible or difficult to perform in a conventional classroom. Because the experiments are provided digitally on the digital table, no or little preparation for the experiments would be required by the teacher. The system and method can deliver and provide new contents by software updates with no additional cost.

The present system and method may also be used for entertainment with educational benefits. The present system and method have a variety of usage in a classroom. The present system and method also provide visual aids using the large display during a lecture. Gamified learning experiences can boost and accelerate students' engagement and comprehension.

For example, the present system and method instantaneously transform the classroom into a science laboratory that is readily prepared to perform any science experiment in no time. The digital science experiments allow students to explore beyond real-world limitations. Using the comprehensive suite of science experiments provided by the present system and method, students can interact with various scientific phenomena across disciplines, gaining insight through interactive and intuitive visualization and hands-on simulation. The digital science experiments are fun and facilitate students' understanding of the principle of the subject. The present system and method can supplement the traditional lectures that are based on textbooks and passively playing multimedia contents.

In another example, the present system and method may be used for a virtual field trip to various locations. The virtual field trip can remove physical limitations and constraints of a traditional field trip.

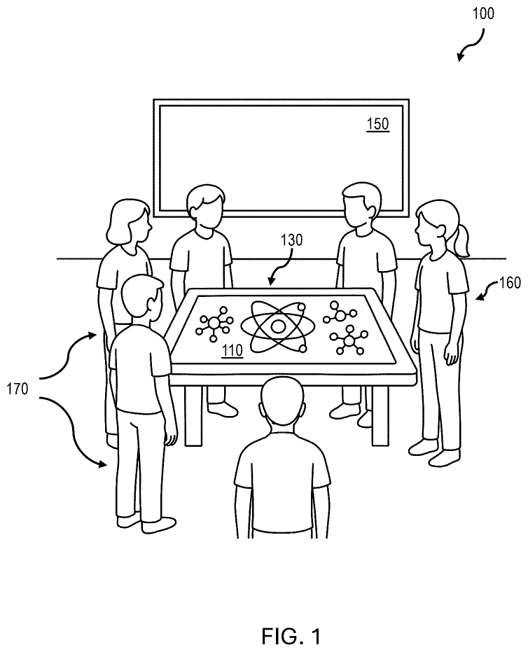

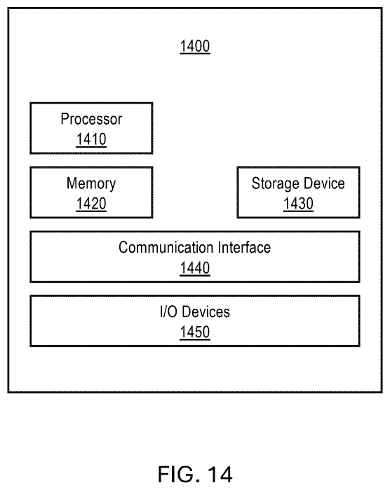

FIG. 1 illustrates an example of a classroom in which educational content is provided by a digital table, according to one embodiment. A classroom 100 includes a digital table 130 that has a display 110. The display 110 may be a flat panel display that is arranged on the top surface of the digital table 130. Although it is not shown, the digital table 130 may further includes a computer (e.g., 1400 of FIG. 14) that is configured to run various digital science experiments. The computer may run on an operating system (e.g., Windows 11) and has a central processing unit (CPU) (Intel Core i7-7800K), a graphics processing unit (GPU) (e.g., NVIDIA Geforce 2070), a random-access memory (RAM), and a hard disk drive (HDD).

The display 110 may be a multi-point (e.g., 4 or more) touch-enabled interactive display that allows students 170 and an instructor 160 to simultaneously interact with the digital table 130. A touch sensor may be incorporated with the display 110 of the digital table 130 or provided separately onto the flat top surface of the display 110. The display 110 may be configured to be positioned horizontally, vertically, lifted, lowered, or positioned at an arbitrary angle depending on a classroom environment and/or a user setting.

The classroom 100 may further include an auxiliary display 150 in the classroom. The contents and experiments that are run on the digital table 130 may be displayed on the display 110 in conjunction with the auxiliary display 150. The auxiliary display 150 may serve as a digital whiteboard by the instructor 160. The instructor 160 may interact with the students 170 while the students 170 perform a science experiment. The instructor 160 may provide guidance, lecture, ask and answer questions, explain the procedures, etc.

The students 170 may perform a digital science experiment under the supervision of the instructor 160. The instructor 160 may or may not be physically present in the classroom 100. In another embodiment, a human instructor 160 may be replaced or augmented with a virtual instructor. The virtual instructor may be a computer-generated character, an Artificial Intelligence (AI)-generated smart figure who can interact and converse with the students 170, or a video of a human instructor who may be connected remotely. The virtual instructor may appear in either the display 110, the auxiliary display 150 or both. The virtual instructor can be pre-configured or programmed to deliver a lecture as well as supervising the students 170 and controlling the contents displayed on the display 110 and/or the auxiliary display 150. A human instructor may collaborate with the virtual instructor during a science experiment. This may greatly save the instructor 160 time and energy to prepare the science experiment. The digital science experiment may be run by the students 170 with no or little intervention or supervision by the instructor 160.

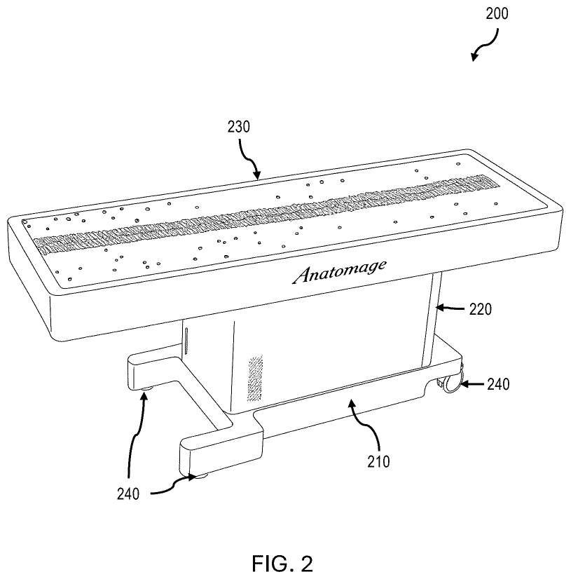

FIG. 2 shows an example of a digital table, according to one embodiment. The digital table 200 may correspond to the digital table 130 of FIG. 1. The digital table 200 includes a base 210, a body 220, and a display 230 that is fixed to the body 220. Although not shown, the digital table 200 may include a computer inside the body 220. The computer is configured to store and run digital science experiments using the display 230. The base 210 may include wheels 240 to move in or around a classroom.

The display 230 may be large enough to be viewed, interacted, and shared by several users. The display 230 may be a high-definition large display (e.g., 84-inch or a similarly sized). The digital table 200 provides a multi-point touch interface. The top surface of the display 230 may be protected by an overlaid tempered glass. The digital table 200 may further integrate various communication interfaces (e.g., Wi-Fi, Bluetooth, HDMI, USB, Ethernet ports) to support seamless connectivity to the network (e.g., Internet, an external database) and I/O devices such as projectors, external monitors/displays, and any other peripheral devices.

The digital table 200 may provide various science experiments that are aligned with instructional units and learning material according to a school curriculum. For example, the science experiments may be provided in instructional modules that are mapped to the middle school (MS) and high school (HS) level science experiments and Next Generation Science Standards (NGSS). The experiments and modules provide a wide range of digital science experiments aligned with specific NGSS codes across various subjects such as biology, chemistry, physics, earth and space science (ESS), and engineering as outlined in Table 1 through Table 8.

TABLE 1
Middle School Physical Science (MS-PS1 to MS-PS4)

| Standard | Subject | Experiments/Modules |
|---|---|---|
| MS-PS1-1 to | Biology, | Cell Membrane Transport, Osmosis, |
| MS-PS1-5 | Chemistry | Calorimetry, Chemical Equilibrium, |
| | | Electrolysis of Water, Formation of Sodium |
| | | Chloride, Gold Plating, Molecular Speed |
| | | Distribution, Reactivity of Metals, |
| | | Replacement Reactions, Formation of |
| | | Sodium Chloride, Oil Refinery, Boiling Point, |
| | | Calorimetry, Ideal Gas Law, Molecular Speed |
| | | Distribution, Oil Refinery, Conservation of |
| | | Mass, Formation of Sodium Chloride |
| MS-PS2-1 to | Physics, | Air Hockey, Collision Force, Viscosity, |
| MS-PS2-5 | Chemistry | Buoyancy, Electromagnetism, Gyroscope, |
| | | Mass and Spring, Moment of Inertia, Newton's |
| | | Second Law of Motion, Orbital Motion, |
| | | Pendulum, Terminal Velocity, Coulomb's Law, |
| | | Electric Circuit, Electromagnetism, Millikan |
| | | Oil Drop Gravitational Acceleration |
| MS-PS3-1 to | Physics, | Mass and Spring, Pendulum, Projectile Motion, |
| MS-PS3-5 | Chemistry | Boiling Point, Ideal Gas Law, Molecular |
| | | Speed DistributionThermal Equilibrium, |
| | | Collision Force |
| MS-PS4-1 to | Physics, | Mass and Spring, Pendulum, Ripple Tank, |
| MS-PS4-3 | Chemistry | Slinky Waves, UV-Visible Spectroscopy, |
| | | Doppler Effect, Optics Table, Rainbow |
| | | Formation, Ripple Tank, Slinky Waves, |
| | | Radio Wave Communication |

TABLE 2
Middle School Life Science (MS-LS1 to MS-LS4)

| Standard | Subject | Experiments/Modules |
|---|---|---|
| MS-LS1-1 to | Biology | Bacteria Explorer, Bacterial Handprint, Cell |
| MS-LS1-6 | | Explorer, Meiosis, Mitosis, Plant Cell Explorer, |
| | | Virtual Cat and Dog, Virtual Microscopy, |
| | | Life of Ants, Virtual Microscopy, Bacterial |
| | | Handprint, Carrying Capacity, Homeostasis and |
| | | Tropism, Respiration vs Photosynthesis, |
| | | Photosynthesis: Light Reactions |
| MS-LS2-1 to | Biology | Biodiversity, Carrying Capacity |
| MS-LS2-4 | | |
| MS-LS3-1 to | Biology | Gene Expression, Human Karyotyping, Fruit |
| MS-LS3-2 | | Fly Genetics, Meiosis, Mitosis |
| MS-LS4-1 to | Biology, | Radioactive Dating, Case Library, Homologous |
| MS-LS4-6 | Chemistry | Structures, Phylogenetics, Virtual Cat and Dog, |
| | | Natural Selection, Artificial Selection, |
| | | Fruit Fly Genetics, Natural Selection |

TABLE 3
Middle School Earth and Space Science (MS-ESS1 to MS-ESS3)

| Standard | Subject | Experiments/Modules |
|---|---|---|
| MS-ESS1-1 to | ESS, | Lunar Phases, Orbital Motion |
| MS-ESS1-3 | Physics | |
| MS-ESS2-2 to | ESS | Plate Tectonics, Stream Table |
| MS-ESS2-3 | | |
| MS-ESS3-1 to | ESS, | Stream Table, Earthquake Detection, Carbon |
| MS-ESS3-5 | Biology | Cycle, Tulare Lake, Earth's Temperature |

TABLE 4
Middle School Engineering and Technology (MS-ETS1)

| Standard | Subject | Experiments/Modules |
|---|---|---|
| MS-ETS1-1 to | Physics, | Biodiversity, Carrying Capacity, Electric |
| MS-ETS1-4 | Biology | Circuit, Fruit Fly Genetics |

TABLE 5
High School Physical Science (HS-PS1 to HS-PS4)

| Standard | Subject | Experiments/Modules |
|---|---|---|
| HS-PS1-1 to | Chemistry, | Acid-Base Titration, Atomic Orbitals, Flame |
| HS-PS1-8 | ESS, | Spectroscopy, Periodic Table, Reactivity |
| | Physics | of Metals, Replacement Reactions, |
| | | Formation of Sodium Chloride, Gold Plating, |
| | | Reactivity of Metals, Replacement |
| | | Reactions, Boiling Point, Capillary Action, |
| | | Gold Plating, Oil Refinery, Plant Pigment |
| | | Chromatography, Viscosity, Calorimetry, |
| | | Electrolysis of Water, Chemical Equilibrium, |
| | | Electrolysis of Water, Reaction Kinetics, |
| | | Gold Plating, Replacement Reactions, |
| | | Conservation of Mass, Replacement |
| | | Reactions, Nuclear Fission, Radioactive |
| | | Dating, The Sun, Cloud Chamber |
| HS-PS2-1 to | Physics, | Buoyancy, Gyroscope, Mass and Spring, |
| HS-PS2-6 | Chemistry, | Moment of Inertia, Newton's Second |
| | ESS | Law of Motion, Pendulum, Projectile |
| | | Motion, Terminal Velocity, Air Hockey, |
| | | Collisio nForce, Black Hole, Coulomb's |
| | | Law, Orbital Motion, Electromagnetism, |
| | | Lithium-Ion Battery, Viscosity |
| HS-PS3-1 to | Physics, | Air Hockey, Mass and Spring, Pendulum, |
| HS-PS3-5 | ESS, | Thermal Equilibrium, Turbofan Jet Engine, |
| | Chemistry | The Sun, Mass and Spring, Pendulum, |
| | | Turbofan Jet Engine, Electrolysis of |
| | | Water, Electromagnetism, Otto Cycle, |
| | | Solar Cell, Turbofan Jet Engine, Otto |
| | | Cycle, Thermal Equilibrium, Turbofan Jet |
| | | Engine, Mass Spectrometer, Capacitor, |
| | | Coulomb's Law, Magnetic Field |
| HS-PS4-1 to | ESS, | Earthquake Detection, Red Shift, Optics |
| HS-PS4-5 | Physics, | Table, Rainbow Formation, Ripple Tank, |
| | Chemistry | Slinky Waves, Radio Wave Communication, |
| | | Flame Spectroscopy, UV-Visible |
| | | Spectroscopy, CMB Radiation, Double-Slit |
| | | Experiment, Optics Table, Speed of Light, |
| | | Radio Wave Communication, Solar Cell |

TABLE 6
High School Life Science (HS-LS1 to HS-LS4)

| Standard | Subject | Experiments/Modules |
|---|---|---|
| HS-LS1-1 to | Physics, | Gene Expression, Case Library, Virtual Cat and |
| HS-LS1-5 | Biology | Dog, Virtual Microscopy, Homeostasis and |
| | | Tropism, Mitosis, Respiration vs Photosynthesis, |
| | | Photosynthesis: Light Reactions |
| HS-LS2-1 to | Biology | Carrying Capacity, Biodiversity, Aerobic vs. |
| HS-LS2-8 | | Anaerobic Respiration, Carbon Cycle, |
| | | Respiration vs Photosynthesis, Photosynthesis: |
| | | Light Reactions, Biodiversity, Life of Ants |
| HS-LS3-1 to | Biology | Artificial Selection, DNA Replication, Fruit Fly |
| HS-LS3-3 | | Genetics, Gene Expression, Human Karyotyping, |
| | | Phylogenetics, Fruit Fly Genetics, Meiosis, |
| HS-LS4-1 to | Biology | Homologous Structures, Phylogenetics, Artificial |
| HS-LS4-5 | | Selection, Fruit Fly Genetics, Natural Selection |

TABLE 7
High School Earth and Space Science (HS-ESS1 to HS-ESS3)

| Standard | Subject | Experiments/Modules |
|---|---|---|
| HS-ESS1-1 to | ESS, | The Sun, Red Shift, CMB Radiation, Lunar |
| HS-ESS1-5 | Physics | Phases, Orbital Motion, Plate Tectonics |
| HS-ESS2-1 to | ESS, | Plate Tectonics, Stream Table, Carbon Cycle |
| HS-ESS2-6 | Biology | |
| HS-ESS3-3 to | ESS, | Carbon Cycle, Tulare Lake, Earth's |
| HS-ESS3-6 | Biology | Temperature |

TABLE 8
High School Engineering and Technology (HS-ETS1)

| Standard | Subject | Experiments/Modules |
|---|---|---|
| HS-ETS1-2 to | Physics, | Carrying Capacity, Nuclear Fission, |
| HS-ETS1-4 | Chemistry, | Collision Force, Oil Refinery |
| | Biology | |

Each module or experiment may be specifically matched to NGSS standards to ensure pedagogical compliance, depth of scientific understanding, and multi-disciplinary applications. Multiple learning modalities including visualizations, digital experiments, interactive models, and simulations may be adapted. The digital table 200 provides enhanced accessibility and interactivity to diverse educational contents by creating flexible and integrated learning environments.

Examples of educational subjects and topics that the digital table 200 can provide include, but not limited to, STEM subjects taught in a class such as biology, chemistry, ESS, physics, and engineering. The digital table 200 provides an immersive and realistic laboratory-based learning platform that empowers students to investigate and apply and understand scientific theories based on digitally constructed interactive models.

According to one embodiment, the digital table 200 provides educational contents, lessons, modules that are grouped together in a plurality of suites. Each suite may include a collection of relevant subjects and topics according to the STEM education curricula. Some interdisciplinary modules or experiments may be provided in more than one suite or grouped together based on their relevance.

The digital table 200 transforms a traditional classroom into an immersive virtual science experiment laboratory. The instant transformation provides not only convenience for the students and teachers but also flexibility and easy accessibility to various science topics while igniting students' curiosity and passion in science. The science experiments may be implemented fully or partially in a software application providing advanced visualization and interactive technology. In some embodiments, a science experiment provides virtual instruments, devices, and apparatuses that the students can interact with and learn to use without literally requiring them. The elimination of physical instruments, devices, and apparatuses that are necessities in a traditional science class can save cost for science education and boosts the teaching and learning experiences to the next level. In particular, the students can engage with the scientific subjects by visual cues and instructions and interactively experience, touch, observe, and toy with the displayed content without requiring a dedicated laboratory and/or complex equipment. The large formfactor of the digital table 200 enables easy collaboration among the participating students, and their group collaboration can be further supplemented using a variety of interactive peripheral tools and devices.

Anatomage Inc. of San Jose, California provides one of a kind digital science table referred to as Science Table™. Anatomage's Science Table is a revolutionary digital laboratory platform that offers on-demand access to complex virtual science experiments and interactive visualizations for various scientific principles and topics. The digital table 200 provides realistic simulations in various science and engineering subjects and topics including, but not limited to, physics, chemistry, biology, ESS, and other educational subjects. Further, the digital table 200 sparks curiosity in STEM education for both educators and students and boosts classroom discussions by providing augmented hands-on experimentation. The digital table 200 transforms challenging science concepts into virtual science experiments that are fun and more engaging.

The digital table 200 also offers a collection of visually dynamic multimedia presentations that are designed to make learning the science subjects and not only exciting, but also more accessible from any location, time, and/or device. The subjects and topics taught by the digital table 200 inspire students and get them excited about learning science topics through virtual experiments, realistic visuals, and action-drive scrolling technology.

According to one embodiment, the digital table 200 provides a controlled environment to perform a science experiment. A student can control and decide when and how to start, trigger, stop, pause, select, interact with the educational contents. Based on the progress of the experiment and/or their understanding of the principle of the experiment, the students can learn at his/her own comfortable pace and repeat the experiments with many different settings and inputs. This promotes the student's engagement and interaction with other students as well as the instructor. The offering of on-demand learning experience by the digital table 200 forms a collaborative platform for the class by allowing more than one student to control the pace and make the progress of the experiment together. In one embodiment, the digital table 200 may be used to administer a quiz, a test, a contest, etc. It is understood that the digital table 200 may be used for other purposes and usages without deviating from the scope of the present disclosure.

According to one embodiment, the digital table 200 provides a suite of biology experiments. The biology experiment suite provides deep exploration of foundational biology concepts and an opportunity to learn laboratory techniques. The biology experiment suite introduces students to ecological balance, genetics, biodiversity, and evolutionary principles through interactive simulations. These subjects may include specialized biology experiments under the categories of “Bioprocesses and Cells,” “Biotechnology and Microscopy,” “Ecology,” “Genetics and Natural Selection,” and “Real-Tissue Cadavers and More.” It is understood that these categories are only examples, and many other categories and experiments are possible without deviating from the scope of the present disclosure.

Under the category of “Bioprocesses and Cells, “Bacteria Explorer” provides visualization and interaction with the external and internal anatomy of bacteria. “Bacterial Handprint” allows students to explore concepts of bacterial growth. “Cell Explorer” provides interaction with a three-dimensional (3D) eukaryotic cell to learn the structure and organelles. “Cell Membrane Transport” experiments with diffusion, passive transport, and active transport. “Enzymatic Reaction and Inhibition” visualizes dihydrofolate reductase converting reactant to product, and compares in the presence of an inhibitor. “Meiosis” divides a cell by meiosis to investigate how cells pass on genetic information. “Mitosis” duplicates a cell through mitosis and explore how cells copy themselves. “Osmosis” experiments with how changing salt concentration affects osmosis in red blood cells. “Photosynthesis: Light Reactions” enters the chloroplast to explore the process of the photosynthesis light reaction. “Plant Cell Explorer” dissects a 3D plant cell to study structure and various organelles. “Respiration vs Photosynthesis” explores two of life's most important chemical reactions using plants and snails.

Under the category of “Biotechnology and Microscopy,” “Aerobic vs. Anaerobic Respiration” investigates how bacteria survive either using aerobic respiration or anaerobic respiration for energy production. “Centrifugation” separates a blood sample into its components using the biotech process of centrifugation. “DNA Replication” participates in the process of DNA replication by activating enzymes and observing their role in making a copy of the DNA. “Gel Electrophoresis” runs gels and uses DNA fingerprinting to search for genetic matches. “Polymerase Chain Reaction” explores how to successfully amplify DNA with a user-controlled thermal cycler. “Virtual Microscopy” controls a virtual microscope with several objective levels and microorganisms to study.

Under the category of “Ecology,” “Biodiversity” investigates how sea stars affect the biodiversity of a tidepool. “Carbon Cycle” activates volcanoes, factories, and photosynthesis to study how they affect carbon in the atmosphere. “Carrying Capacity” varies environmental resources to determine the effect on rabbit population growth. “Homeostasis and Tropism” changes water and light sources to affect the growth of plants. “Life of Ants” studies the behavior of ants as they interact with different things in their environment.

Under the category of “Genetics and Natural Selection,” “Artificial Selection” experiments with plant genetics by artificially selecting favorable traits to create a harvest of a desired variety. “Fruit Fly Genetics” Investigates the inheritance of eye color, body color, and wing shape traits. “Gene Expression” transcribes and translates a segment of DNA, following its path from a genetic code to a protein. “Human Karyotyping” allows students to sort chromosomes into a karyogram to compare a healthy female and male sample with many different genetic disorders. “Natural Selection” acts as a predator to explore natural selection in moths. “Phylogenetics” investigate the evolutionary history of different species by using a phylogenetic tree, inherited traits, and genetic information.

Under the category of “Real-Tissue Cadavers and More,” “Case Library” provides investigation of CT scans of a variety of mammals, reptiles, birds, fish, etc. “Homologous Structures” rotates and compares the anatomy of different animal limbs in 3D. “Virtual Cat” dissects a real-tissue cadaver for hands-on study of cat anatomy. “Virtual Dog” dissects and explore over 1,100 anatomical structures reconstructed from a real canine cadaver.

According to one embodiment, the digital table 200 provides a suite of chemistry experiments. The chemistry experiment suite focuses on atoms, molecular structures, reactions, and lab techniques. These subjects may include specialized chemistry experiments under the categories of “Atoms, Molecules, and Elements,” “Lab Techniques and Applications,” and “Reactions and Applications.” It is understood that these categories are only examples, and many other categories and experiments are possible without deviating from the scope of the present disclosure.

Under the category of “Atoms, Molecules, and Elements,” “Atomic Orbitals” builds 14 different s, p, d and f orbitals by random sampling of an electron's probability to exist at a certain location. “Hybrid Orbitals” combines atomic orbitals to create hybrid orbitals and explore the basis of molecular shapes.

“Molecular Geometry” builds atoms to observe the variation in molecular geometry. “Molecule Visualizer” interacts with 3D molecules from a library of commonly discussed compounds in chemistry and biology. “Periodic Table” provides 3D interactive periodic table to learn categories of elements, orbital blocks, phases, etc.

Under the category of “Lab Techniques and Applications,” “Boiling Point” conducts an experiment to determine the boiling points of different liquids. “Calorimetry” explores heat of solution by predicting and measuring temperature change with a series of dissolved salts. “Capillary Action” explores the adhesion and cohesive forces of substances by testing the capillary actions of different liquids. “Flame Spectroscopy” uses a spectroscope to identify an unknown metal based on its spectrum emissions. “Ideal Gas Law” experiments with changes in volume, temperature, and pressure of gasses. “Mass Spectrometer” utilizes a mass spectrometer to test for the relative abundances of each element's isotopes. “Molecular Speed Distribution” uses an experimental apparatus to test how gas speed distributions change with temperature. “Oil Refinery” distills and separates various components of crude oil in an oil refinery. “Plant Pigment Chromatography” separates a mixture of plant pigments by controlling the mobile phase solvent used. “UV-Visible Spectroscopy” explores the fundamentals of spectroscopy with a user-controlled spectrometer. “Viscosity” drops objects in different liquids to observe how viscosity affects the falling speed.

Under the category of “Reactions and Applications,” “Acid-Base Titration” experiments with titrating different acids that have a range of different strengths. “Chemical Equilibrium” controls temperature and volume changes to shift equilibrium in a chemical reaction. “Conservation of Mass” visualizes how the vinegar and baking soda reaction converts reactants to products as mass is conserved. “Electrolysis of Water” experiments with driving chemical reactions with the use of electrolysis. “Formation of Sodium Chloride” reacts sodium and chlorine gas with molecular visualization to study electron transfer and ionic formations. “Gold Plating” explores electrochemistry by attempting gold plating with various solvents and on different metals. “Lithium-Ion Battery” visualizes how a Li-ion battery functions in charging and discharging states. “Nuclear Fission” shoots neutrons to initiate nuclear fission in uranium and visualize chain reactions. “Radioactive Dating” digs up fossils and use radioactive dating methods to identify the age of the fossils. “Reaction Kinetics” explores reaction rate with the reaction of calcium carbonate with hydrochloric acid. “Reactivity of Metals” explores the explosive side of chemistry by observing how metals react with water and acid. “Replacement Reactions” conducts single and double replacement reactions by combining ‘n’ elemental and ‘m’ compound substances.

According to one embodiment, the digital table 200 provides a suite of ESS experiments. The ESS experiment suite may include modules that simulate environmental and astronomical phenomena. These subjects may include specialized ESS experiments under the categories of “Environmental Science and Geology” and “Space.” It is understood that these categories are only examples, and many other categories and experiments are possible without deviating from the scope of the present disclosure.

Under the category of “Environmental Science and Geology,” “Earth's Temperature” observes how global temperatures have changed over the last 150 years due to human activity. “Earthquake Detection” determines the location of earthquakes by using seismometers and triangulation. “Plate Tectonics” journeys through time to witness the movement of Earth's tectonic plates. “Stream Table” simulates the processes of erosion, sediment transport, and deposition that occur in natural water systems, such as rivers and streams. “Tulare Lake” explores the history, environmental impact, and hydrology of Tulare Lake.

Under the category of “Space,” “Black Hole” travels towards a black hole to experience the gravitational pull and time dilation caused by massive objects in space. “Lunar Phases” observes the moon's phases from any location on Earth or from space. “Red Shift” searches for galaxies in the night sky and observe the shift in light from those galaxies. “The Sun” manipulates time to witness the life cycle of the Sun.

According to one embodiment, the digital table 200 provides a suite of physics experiments. The physics experiment suite includes interactive modules that investigate fundamental physical laws and phenomena. These subjects may include specialized physics experiments under the categories of “Circuits and Charges,” “Motions and Collisions,” “Properties of Light,” “Subatomic”, “Thermodynamics,” and “Waves and Fields.” It is understood that these categories are only examples, and many other categories and experiments are possible without deviating from the scope of the present disclosure.

Under the category of “Circuits and Charges,” “Capacitor” designs a capacitor to affect the amount of charge and energy stored. “Coulomb's Law” performs an experiment to reveal the relationship between charge, distance, and electrostatic force. “Electric Circuit” constructs and explores the behavior of circuits. “Electromagnetism” investigates the connection between electric and magnetic fields by controlling generators and motors. “Millikan Oil Drop” determines the charge of an electron using an oil drop experimental apparatus.

Under the category of “Motions and Collisions,” “Air Hockey” plays on an air hockey table to explore conservation of momentum in collisions. “Buoyancy” pilots a submersible as one controls the buoyancy and direction of a craft to find underwater treasure. “Collision Force” crash test different cars to study the physics of car design. “Gyroscope” experiments with the properties of gyroscopic movement and rotational inertia. “Mass and Spring” experiments with damped oscillations of various masses and springs. “Moment of Inertia” races objects down an incline to observe how different shapes have different velocities due to rotational inertia. “Newton's Second Law of Motion” proves the relationship between force, mass, and acceleration using masses and an air track. “Orbital Motion” explores orbital motion of the Sun-Earth-Moon system and construct your own orbits. “Pendulum” experiments with simple harmonic motion of pendulums with various masses and string lengths. “Projectile Motion” launches cannonballs with control of launch angle and speed to attempt hitting target boxes. “Terminal Velocity” drops objects off of a tall building to observe their terminal velocity.

Under the category of “Properties of Light,” “Double-Slit Experiment” investigates the wavelike properties of light by passing it through single and double-slits. “Optics Table” uses different sources of light and various glass optics to explore refraction and dispersion of light. “Rainbow Formation” shows how a rainbow is formed from the light reflecting inside water droplets. “Solar Cell” determines the optimal position of a solar cell for energy production. “Speed of Light” replicates Fizeau's experiment to determine the speed of light.

Under the category of “Subatomic”, “Cloud Chamber” creates and visualizes radiation to explore radioactive decay and subatomic particles.

Under the category of “Thermodynamics,” “Otto Cycle” studies the sequence of a four-stroke engine and its relationship to PV and TS diagrams. “Thermal Equilibrium” heats up and cools down different objects, exploring the physics of heat transfer. “Turbofan Jet Engine” explores the inner working of a Turbofan Jet Engine.

Under the category of “Waves and Fields,” “CMB Radiation” explores the cosmic microwave background (CMB) radiation of our universe. “Doppler Effect” measures how a pitch of an ambulance siren changes as it drives by. “Gravitational Acceleration” compares gravitational acceleration on the Earth, Moon, Sun, and in vacuum tubes by dropping a bowling ball and feather. “Magnetic Field” visualizes the magnetic fields created by bar magnets and how multiple fields interact. “Radio Wave Communication” sends Morse code messages using the technology of AM and FM radio wave communication. “Ripple Tank” generates waves in water to explore amplitude, frequency, and interference of waves. “Slinky Waves” takes control of a slinky to study the properties of waves such as frequency, amplitude, and speed.

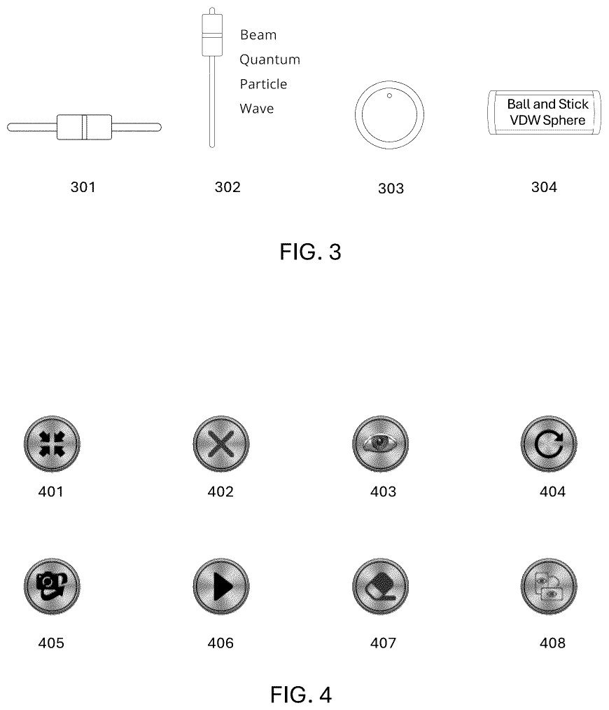

FIG. 3 shows examples of user interface controls usable in a digital experiment, according to one embodiment. The control 301 is a slider that can be dragged with one finger. The control 302 is a mode selector that can be dragged with one finger. Although the control 302 shows four modes of beam, quantum, particle, and wave, it is understood that the number and labels of the modes may vary according to the experiment. The control 303 is a dial that can be dragged with one finger in an upward motion to increase or downward motion to decrease a value. The control 304 is a scroll selector that can be dragged with one finger in an upward or downward motion. Although the control 304 shows an example selection labelled “Ball and Stick VDW Sphere,” it is understood that the number and labels of the selections of the control 304 may vary based on the experiment.

FIG. 4 shows examples of buttons usable in a digital experiment, according to one embodiment. The button 401 can be tapped to minimize a scene. The button 402 can be tapped to exit a scene. The button 403 can be tapped to turn on an “Analysis Mode.” The button 404 can be tapped to reset a scene. The button 405 can be tapped to reset a camera changing a perspective of a scene. The button 406 can be tapped to turn on “Play.” The button 407 can be tapped to erase a graph. The button 407 can be tapped to rotate a scene.

The science experiments provided by the digital table 200 provides unique teaching, learning, and training experiences and features. According to one embodiment, teacher guides provide an instructor with a consistent and structured format for the experiments to ensure clarity and usability of the experiments. The teacher guides can provide the instructor with a unique classroom teaching experience, whether the instructor is in person or remote from the classroom.

The instructor may refer to a teacher guide to effectively introduce and guide the students through the corresponding experiment. In one embodiment, the teacher guide may be provided as a computer application that can run on the same computer that runs the experiment on the digital table or remotely from the digital table. In the latter case, the teach guide application may connect to the experiment. The teach guide may allow the instructor to intervene or control the experiment real-time while monitoring a pace or progress of the experiment as well as the students' activities (e.g., clicking on controls, buttons, etc.) during the experiment. The teach guide may also be used as a tool to assess the students' performance on the experiment based on their activity, pace, completion of a given quiz, mission, or a task, etc.

The teach guide also provides an overview that outlines the overall purpose of the experiment and highlights the key topics that the students can explore. Learning goals contained in the overview focus on specific skills and knowledge that the students can develop through the experiment. Students' activities and level of understanding can be checked against the learning goals, and the instructor can encourage them to think critically about the concepts that they observe during the experiment. Questions as outlined in the teacher guide can promote the exploration of advanced topics related to the experiment to develop a deeper understanding of the concepts. The teach guide can further incorporate recommendations on how the experiment can be used to teach a variety of topics. Many scenes are multifaceted and can revisited to teach different topics, allowing for extended use across multiple lessons.

The teach guide may also include a visuals and interactions section that provides labeled photos of the experiment scenes and a detailed list of its interactive components. The instructor may use the visuals and interactions section to explain the function of each component and provide guidance on how to interact with them effectively.

The teach guide may also include a procedure section that is a step-by-step guide for conducting the experiment. While experiments can often be explored in various ways, the streamlined process of the procedure section shows the key features of the experiment.

The teach guide may also provide an analysis mode in an experiment. The analysis mode may be activated by tapping on the “eye icon” button (e.g., eye icon 403 of FIG. 4).

The analysis mode reveals additional visuals and information that cannot typically be observed in a traditional experiment. Each analysis mode is unique for its respective experiment, so this section explains its specific function and purpose.

The teacher guide may guide the instructor regarding the alignment of the experiment with the NGSS standard such as performance expectations, disciplinary core ideas, science and engineering practices, cross-cutting concepts, connections to the nature of science and to engineering, technology, and connection to applications of science.

Among the many biology experiments that are provided by the digital table 200, “Fruit Fly Genetics” experiment allows a student to virtually breed fruit flies to discover how dominant and recessive alleles shape the observable traits of the fruit flies. Alike many other experiments, this type of experiment is impossible to perform in a conventional classroom environment due to many constraints and limitations, for example, the time required to breed the fruit flies. The experiment can be started, stopped, paused, controlled, and repeated with various of combinations of inputs by the student without constraints and limitations, and the digital table 200 provides accelerated or decelerated visualization of a science experiment that is otherwise impossible in a conventional classroom setting. In this experiment, the student can explore genetic inheritance by breeding fruit flies and observing their phenotypes and genotypes. By selecting specific flies to reproduce, they can study Mendelian genetics and analyze how dominant, recessive, and sex-linked traits manifest in offspring. The scene includes tools to compare phenotypic ratios, visualize genetic crosses using Punnett squares, and investigate the genetic mechanisms behind eye color, body color, and wing shape. The Fruit Fly Genetics experiment integrates seamlessly into units on genetics or evolution, providing a hands-on approach to understanding key biological principles.

The Fruit Fly Genetics experiment can teach the students to compare and contrast phenotypes and genotypes by visually inspecting flies and viewing their underlying alleles, observe how dominant and recessive alleles produce various phenotypes, determine how various crosses (using either homozygous and/or heterozygous parent flies) can result in specific offspring genotype and phenotype ratios using Mendelian genetics and Punnett squares, and identify the difference in phenotypic ratios when traits are sex-linked versus non-sex-linked.

Examples of the exploration questions that can be asked to the students in the Fruit Fly Genetics experiment are, but not limited to, which phenotypes of eye color, body color, and wing shape result from the dominant alleles, and how can you tell by looking at both homozygous and heterozygous flies for each trait?, why do the male flies have only one allele for eye color while the female flies have two, and what does this tell you about the chromosome that contains the allele?, what are the expected ratios for the F1 breeding population when crossing a heterozygous male with a heterozygous female in either body color or wing shape, do the experimental results support the expected ratio, what does this tell you about the chromosome(s) that these traits are located on?, and are eye color ratios the same or different than those found for body color and wing shape, and what does this tell you about the chromosome(s) that this trait is located on?

The Fruit Fly Genetics experiment can be used as an activity during a Genetics Unit for students to experiment with Mendelian and Sex-linked inheritance patterns. The Fruit Fly Genetics experiment can also be used as an activity during a Natural Selection Unit for students to experiment with how the frequency of traits can change based on who survives and reproduces each generation.

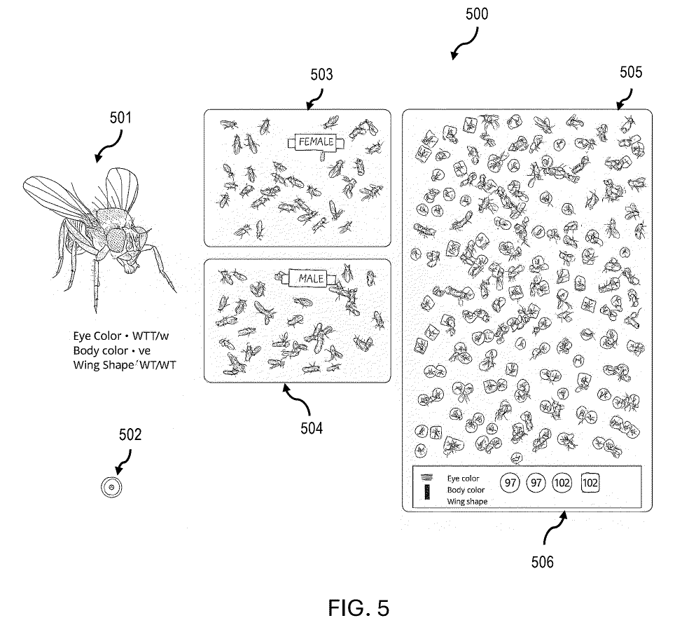

FIG. 5 shows an example scene of the Fruit Fly Genetics experiment, according to one embodiment. The experiment 500 shows a fruit fly 501 that is currently selected. The selected fruit fly 501 may be shown as enlarged in an investigation window. The fruit fly 501 may have specific phenotypes such as an eye color, a body color, a wing shape, etc. The button 502 may be tapped to reset the user's selection and/or reset the ongoing experiment. Male and female fruit flies are initially separated in the population areas 503 and 504 prior to starting the experiment. The user can select any fruit fly from the female population area 503 and the male population area 504 at any time during the experiment and drag it to the breeding area 505. As the breeding progresses, the number of the fruit flies multiplies in the breeding area 505, and the statistical data may be shown and updated in the legend 506 based on their eye color, body color, and wing shape, etc.

The examples of the available items and the user's action in the Fruit Fly Genetics experiment are summarized in Table 9. Each item or action serves to perform an action as outlined in the Purpose column corresponding to the user's interaction.

TABLE 9
Visuals and Interactions for Fruit Fly Genetics experiment

| Item/Action | Purpose | User Interaction |
|---|---|---|
| Fruit Flies | Examine and | Tap with one finger/Tap |
| | reproduce | and drag with one finger |
| Analysis Mode Button | Show the genotype | Tap with one finger |
| (investigate window) | of the chosen fly | |
| Breeding Window | Allow flies to | Tap and drag fly into |
| | reproduce | the area |
| Analysis Mode Button | Show the phenotypes | Tap with one finger |
| (breeding window) | of offspring | |
| | generation | |
| Trait Mode Selector | Switch between which | Tap and drag with one |
| | phenotype is being | finger |
| | shown (in breeding | |
| | analysis mode) | |
| Breeding Window Top | Protect flies from | Tap and drag fly into |
| Left Box | spray | the area |
| Spray Bottle Button | Remove flies from the | Tap with one finger |
| | breeding area | |
| Reset Button | Reset the scene back | |
| | to its initial state | |

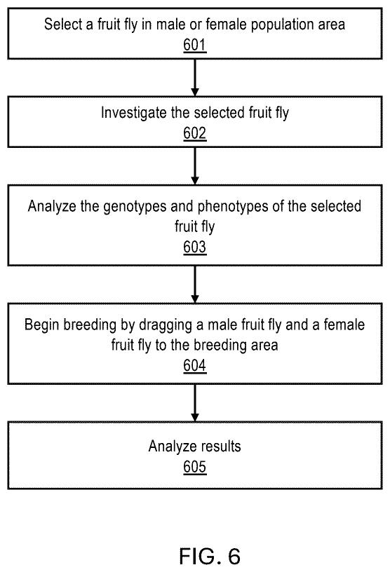

FIG. 6 shows an example procedure of the Fruit Fly Genetics experiment, according to one embodiment. A user selects a fruit fly by tapping on in a female population area or a male population area (601). The selected fruit fly may appear enlarged in an investigation window and may be shown with a changed color (e.g., red) or glow in a specific color (602). The user may turn on an analysis mode by tapping on the Analysis Mode button (e.g., eye icon 403 of FIG. 4) to further analyze the genotypes and/or phenotypes of the selected fly (603). After the user selected and dragged at least one male fruit fly and at least one female fruit fly to the breeding area, the breeding starts (604). The user waits for a stopwatch to complete the breeding experiment (605). The time for completing the breeding experiment can be predetermined or changed to accelerate or decelerate the experiment based on a user setting. After completion, the user can analyze the results by tapping on the Analysis Mode button in the breeding area to view the traits of the offspring population (606). For example, circles may represent females, and squares represent males. The color of the first shape is the dominant trait, and the color of the second shape is the recessive trait. The user can select a mode by tapping on the Trait Mode Selector button (e.g., mode selector 302 of FIG. 2) to toggle between the traits.

The user may continue, reset or restart breeding another generation by tapping and dragging any male and female fruit flies. The user may keep for breeding to a partial area (e.g., the top left corner) of the breeding area. In one embodiment, as the breeding progresses, another fruit flies may be introduced to the breeding area. The Spray Bottle button may be used remove the unwanted population from the breeding area. Another experiment may be run by repeating the steps of 601 through 605.

According to one embodiment, the Fruit Fly Genetics experiment may provide two Analysis Mode Buttons, one in the investigation window and one in the breeding window. The user may tap on the first Analysis Mode button in the investigation window to view the selected flies' genotypes for eye color, body color, wing shape, etc. The second Analysis

Mode button in the breeding window may show the distribution of the phenotypes in the offspring. The user may use the Mode Selector to look at the phenotype distribution of the traits. For example, for the eye color, orange represents the dominant red eyes while gray represents the recessive white eyes; for the body color, brown represents the dominant brown body while gray represents the recessive ebony body; for the wing shape, gray represents the dominant full-size wings while orange represents the recessive vestigial wings.

Among the many chemistry experiments that are provided by the digital table 200, “Mass Spectrometer” experiment provides a virtual mass spectrometer that can be used for a student to analyze the isotopic composition of various elements, for example, magnesium, copper, lead, zirconium, carbon, and boron. The Mass Spectrometer experiment shows ions that are bent through a magnetic field allowing the student to explore the inner workings of a mass spectrometer to uncover the hidden isotopes of elements. By ionizing the metal samples and adjusting the strength of the magnetic field, the student can guide ions through the spectrometer and detect their mass-to-charge ratios. A bar graph displays the relative abundance of isotopes providing insight into their natural distribution. The Mass Spectrometer experiment integrates seamlessly into units on atomic structure, nuclear chemistry, or electromagnetism, offering a hands-on approach to understanding isotopes and the applications of mass spectrometry.

The Mass Spectrometer experiment can teach the students to identify the isotopes of six elements and their relative abundances using a mass spectrometer, and explains how a magnetic field bends ion paths and separates them based on their mass-to-charge ratio. The student learns how to use a mass spectrometer to measure isotope masses and interpret bar graph data.

Examples of the exploration questions that can be asked to the students in the Mass Spectrometer experiment are, but not limited to, what are the isotopes of the six elements tested in this lab, and what are their relative abundances?, how did the strength of the magnetic field affect ions of different elements, and can you use the same magnetic field strength on all six elements, and if you were given an unknown element to test, how could you determine what element it is?

The Mass Spectrometer experiment can be used as an activity during an Atoms, Isotopes, and Ions Unit to learn about atomic isotopes and their natural frequency. The Mass Spectrometer experiment can be used as a teaching tool during an Electromagnetics Unit to demonstrate how magnetic fields can affect particles, and a Nuclear Chemistry Unit to show the isotopes of different elements.

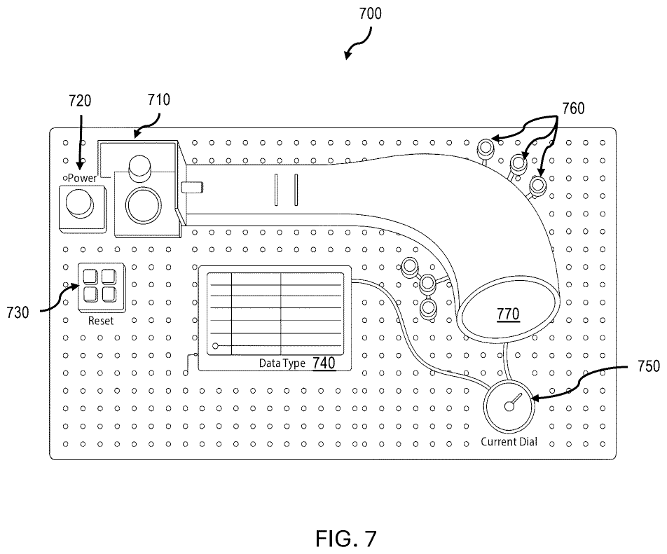

FIG. 7 shows an example scene of the Mass Spectrometer experiment, according to one embodiment. The experiment 700 includes an ionization chamber 710, a power button 720, a reset button 730, a bar graph 740, a current dial 750, a magnetic coil 760, and a detector 770. Although it is not explicitly shown in FIG. 7, the experiment 700 may further include other buttons and instruments, for example, an analysis mode button.

The examples of the available items and the user's action in the Mass Spectrometer experiment are summarized in Table 10. Each item or action serves to perform an action as outlined in the Purpose column corresponding to the user's interaction.

TABLE 10
Visuals and Interactions for Mass Spectrometer experiment

| Item/Action | Purpose | User Interaction |
|---|---|---|
| Reset Button | Reset the scene back to its | Tap with one finger |
| | initial state | |
| Analysis Mode | Show the magnetic field | Tap with one finger |
| Button | lines in blue and the current | |
| | direction in green | |
| Metal Blocks | Metal samples to place into | Drag with one finger |
| | the ionization chamber | |
| Power Button | Turn on/off the laser | Tap with one finger |
| Current Dial | Adjust the magnetic field by | Turn clockwise or |
| | increases/decreasing the | counterclockwise with |
| | current | one finger |
| Data Type Button | Toggle the bar graph from | Tap with one finger |
| | relative abundance to | |
| | abundance | |

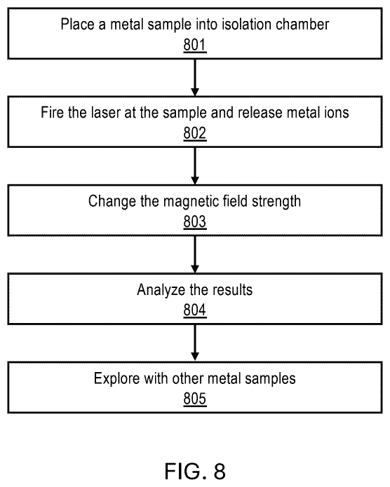

FIG. 8 shows an example procedure of the Mass Spectrometer experiment, according to one embodiment. A user selects and places a metal sample into the ionization chamber by tapping and dragging with a single finger (801). A list of metal samples may be provided to the user. A laser is fired at the metal sample that is placed in the ionization chamber by pressing the power button, and metal ions are released (802). The user can change the strength of the magnetic field formed by the magnetic coil by turning the current dial (e.g., 750 of FIG. 7), and adjust the magnetic field to have the released metal ions reach detected by the detector (803). The user can analyze the results (804). The data of the results may be shown in the bar graph (e.g., 740 in FIG. 7). The type of the collected data and graph may be toggled by tapping a toggle button. The experiment may repeat with another metal samples (805). The user may tap on the Analysis Mode button (e.g., eye icon 403 of FIG. 4) to show the magnetic field in the bend of the spectrometer's tunnel.

Among the many physics experiments that are provided by the digital table 200,

“Electric Circuit” experiment allows a student to learn how voltage, current, and resistance interact within electric systems. The student can construct electric circuits by arranging batteries, resistors, lightbulbs, fans, and switches on a virtual breadboard. Using a multimeter, the student can measure voltage and current across different circuit elements to observe how various configurations affect electrical flow. The Electric Circuit experiment allows the student to explore both series and parallel circuits, apply Ohm's and Kirchhoff's laws, and analyze how changes in resistance and voltage impact current. The Electric Circuit experiment also integrates well into units on electricity, physics, or engineering, providing a hands-on approach to understanding how circuit components interact.

The Electric Circuit experiment can teach the student to identify the relationship between circuit elements and their effect on current and voltage, analyze the behavior of a circuit in both series and parallel configurations, and evaluate a circuit's voltage and current by applying Ohm's law and Kirchoff's law, and observe the flow of electrons and determine whether the circuit is open or closed.

Examples of the exploration questions that can be asked to the students in the Electric Circuit experiment are, but not limited to, how does the resistance of a resistor impact the flow of electricity in a circuit?, how does the configuration of resistors in a circuit, such as series or parallel, affect the combined resistance of the resistors? (Hint: Apply Ohm's law to series and parallel circuits.), how do switches impact the flow of electricity in a circuit?, when the switch is flipped on, is the circuit open or closed?, and how can we measure the amount of power flowing through a circuit element? [Hint: Power (watts)=Current (amps) x Voltage (volts)].

The Electric Circuit experiment can be used as an activity during an Electric Circuits Unit to experiment with building different types of circuits and learn to solve for voltage, current, and resistance, as a teaching tool during an Electric Circuits Unit to demonstrate different types of circuits that can be built, as an activity during an Engineering Unit to experiment with designing electric circuits for different purposes.

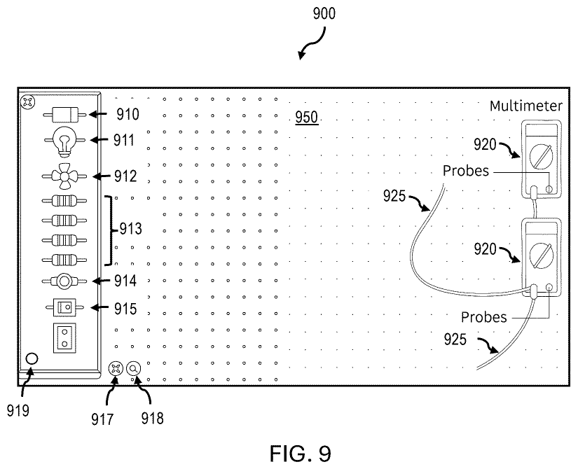

FIG. 9 shows an example scene of the Electric Circuit experiment, according to one embodiment. The experiment 900 includes a battery 910, a light bulb 911, a fan 912, a plurality of resistors 913, a rotating switch 914, an on/off switch 915, a pencil button 917, an eraser 918, an eraser button 918, a reset switch 919, one or more multimeters 920, a plurality of probes 925, and a breadboard 950.

The examples of the available items and the user's action in the Electric Circuit experiment are summarized in Table 11. Each item or action serves to perform an action as outlined in the Purpose column corresponding to the user's interaction.

TABLE 11
Visuals and Interactions for Electric Circuit experiment

| Item/Action | Purpose | User Interaction |
|---|---|---|
| Reset Button | Reset the scene back | Tap with one finger |
| | to its initial state | |
| Pencil Button | Place wires on the | Tap and drag with one finger |
| | breadboard | |
| Eraser Button | Remove wires from | Tap and drag with one finger |
| | the breadboard | |
| Battery (1.5 V) | Power the circuit | Tap and drag with one finger to |
| Light Bulb | An indicator that | place |
| (1 ohm) | the circuit is | |
| Fan (1 ohm) | correctly completed | Tap with one finger to rotate |
| Resistors | Increase the resistance | |
| | along the wire and | |
| | reduce the current | |
| Rotating | Switch between two | |
| Switch | paths of the circuit | |
| On/Off Switch | Turn on/off the | Tap and drag with one finger |
| | circuit | Tap with one finger to open and |
| | | close the switch |
| Multimeters | Measure the voltage | Tap and drag with one finger to |
| | or current across | move the multimeter |
| | a circuit component | Tap and drag with one finger to |
| | | move probes |
| | | Tap with one finger to switch |
| | | the dial from volts to amps |

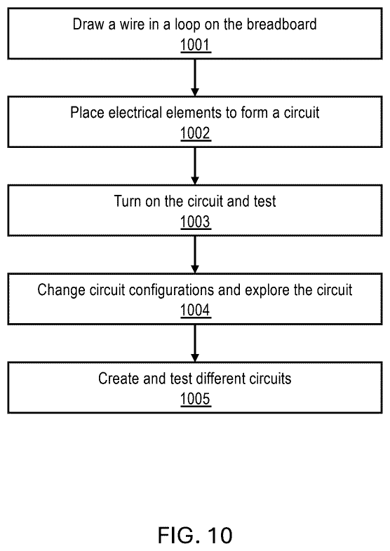

FIG. 10 shows an example procedure of the Electric Circuit experiment, according to one embodiment. A user draws a wire in a loop on the breadboard using a pencil tool (e.g., using the pencil button 917 of FIG. 9) and dragging a finger around the breadboard (1001). The pencil tool may be deactivated before continuing to the next steps. The user then places a battery on the wire drawn using the pencil tool and further places a fan and a light bulb to form a circuit, then places a resistor and an on/off switch to complete the circuit (1002). After the circuit is complete, the user can turn the circuit on/off and test the circuit (1003). The user may change circuit configurations and explore the changes made to the circuit (1004). For example, the user may create two or more paths in the circuit by placing a rotating switch at an intersection of the paths and explore the circuit by rotating the rotating switch. The user can explore different circuit configurations and test and observe the circuit (1005).

Among the many ESS experiments that are provided by the digital table 200, “Black Hole” experiment allows a student to embark on a journey through space and experience the intense gravity and time dilation of a black hole. In this experiment, the student can pilot a spaceship near a black hole, exploring the effects of its gravitational field and time dilation. As the student travel closer or orbit the black hole, they can observe how mass, distance, and gravity interact, and how time slows down relative to Earth. The user can turn on the Analysis Mode to track key variables like the strength of the gravitational field, time differences, and speed, while visualizing the warping of spacetime. By controlling a virtual spaceship, the student can gain a deeper understanding of how black holes affect both gravity and time, drawing from principles of general relativity.

The Black Hole experiment can teach the student how to observe the effects large astronomical bodies have on gravitational field strength and time, explain how mass and distance influence gravitational field strength, how to interpret data to compare the gravitational field and time dilation at varying distances from the black hole.

Examples of the exploration questions that can be asked to the students in the Black Hole experiment are, but not limited to, what factors can cause the gravitation field strength to increase?, based on this, why does the gravity value increase when you travel toward the black hole but not when you travel in an orbit around the black hole?, what happened to the time on the ship compared to the time on Earth as you get closer to the black hole?, why does this occur?, why do we see a ring of light around a black hole but no light inside of a black hole?

The Black Hole experiment can be used as an activity during a Gravitation Unit to explore how mass and distance affect the gravitational field strength, as a teaching tool during an Astronomy Unit to show the characteristics of a black hole, and as an activity during a Relativity Unit to investigate how spacetime can warp and time dilation occurs.

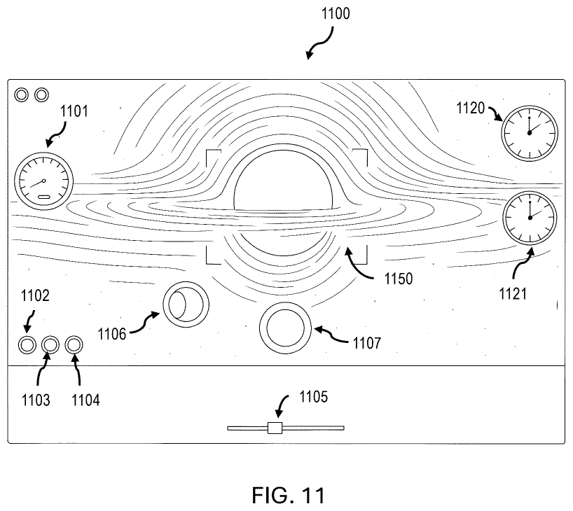

FIG. 11 shows an example scene of the Black Hole experiment, according to one embodiment. The experiment 1100 includes a black hole 1150 and several navigation tools such as a speedometer 1101, a reset button 1102, an autopilot 1103, an analysis mode button 1104, and a speed slider 1105. In addition, a pilot joystick 1106 and a camera joystick 1107 are shown to help the user to pilot the spaceship. A spaceship clock 1210 and the Earth clock 1121 are shown on the right side of the experiment 1100.

The examples of the available items and the user's action in the Black Hole experiment are summarized in Table 12. Each item or action serves to perform an action as outlined in the Purpose column corresponding to the user's interaction.

TABLE 12
Visuals and Interactions for Black Hole experiment

| Item/Action | Purpose | User Interaction |
|---|---|---|
| Reset Button | Reset the scene back to its | Tap with one finger |
| | initial state | |
| Autopilot | Enable autopilot on the | |
| Button | spaceship and orbit around | |
| | the black hole at a constant | |
| | distance | |
| Analysis Mode | Turn on to show a | |
| Button | speedometer for the ship, | |
| | mass, distance, and strength | |
| | of the gravitational field, and | |
| | clocks for observers on the | |
| | ship versus the Earth | |
| Speed Slider | Change the speed of the | Drag left or right with |
| | spaceship | one finger |
| Left Virtual | Change the direction of the | With autopilot off, |
| Joystick | spaceship | tap near the middle |
| Right Virtual | Change the perspective of | of the screen and drag |
| Joystick | the camera | in all directions with |
| | | one finger |

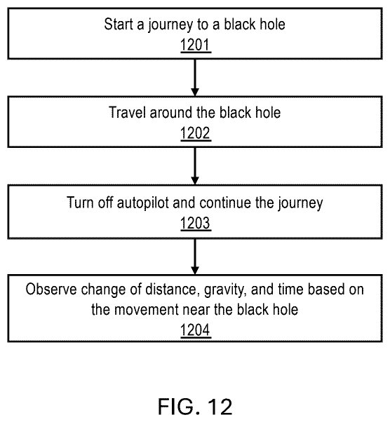

FIG. 12 shows an example procedure of the Black Hole experiment, according to one embodiment. A user may start a journey to a black hole via a spaceship (1201). By default, for example, at the beginning of the journey, an analysis mode may be turned on or can be manually turned on by tapping on the Analysis Mode button. Similarly, an autopilot may be turned on or off by tapping the autopilot button. The virtual spaceship travels around the black hole (1202). The user observes the gravitational field strength and change in time. At any time during the journey, the user may take over the control of the spaceship by turning off the autopilot and start piloting the spaceship manually towards the black hole (1203). The user can observe how gravity and time change as the user pilots the spaceship (1204). For example, the Analysis Mode that is turned on by tapping on the analysis mode button (e.g., eye icon 403 of FIG. 4) shows the mass, distance, and gravitational field strength of the black hole. The distance and gravity values will change as the spaceship flies toward or away from the black hole. The speedometer may be shown to indicate how fast the spaceship is flying. The speed of the spaceship can be changed with the speed slider. The two clocks labeled “Ship” and “Earth” may appear on the right side of the scene. As approaching the black hole, the user can see the difference in the passage of time on the spaceship compared to on Earth.

The experiments and contents described herein may be related to educational contents provided in a digital table platform as shown in FIG. 1 and FIG. 2. In another embodiment, a wide range of interactive contents including the educational contents may be provided in an immersive experience platform.

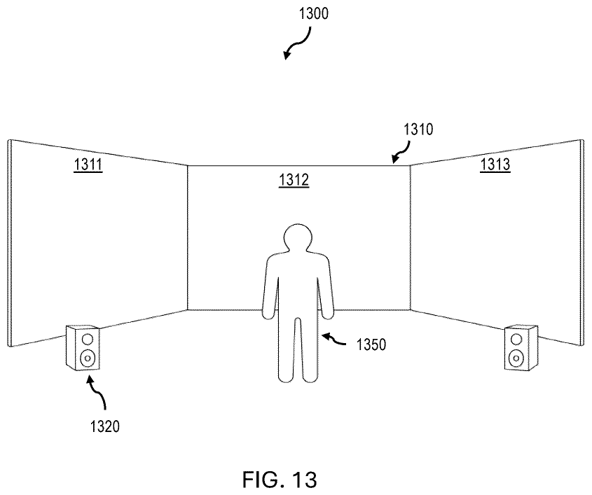

FIG. 13 shows an immersive system for providing interactive content, according to another embodiment. The immersive system 1300 includes a surround screen 1310, an audio system 1320, and a computer (not shown), and a plurality of input devices such as a remote control, a joystick, a ring, a virtual reality (VR) device, a microphone, a keyboard, a voice recognition device, a camera, a touchscreen, a trackpad, mouse, a game controller, a foot pedal, etc. The surround screen 1310 may include an array of high-resolution displays that are arranged horizontally to provide a full-room experience in a room. In one embodiment, the surround screen 1310 includes a first display 1311 that covers the left side of the room, a second display 1312 that covers the front side of the room, and a third display 1313 that covers the right side of the room. However, it is understood that other numbers of displays and arrangement is possible without deviating the scope of the present disclosure.

In one embodiment, the resolution of the surround screen 1310 may be 7,680 by 1,080, 3,840 by 1,080, etc. depending on the configuration. The aspect ratio may be 64:9 or 32:9, etc. depending on the configuration. The audio system 1320 may be a multi-channel surround sound audio system including one or more subwoofer, one or more main speakers, one or more surround speakers, etc. The displays 1311, 1312, and 1313 are wide and tall enough to provide a fully immersive experience to a user 1350 who may be standing in the middle of the room. The user 1350 may move within the room, and the immersive system 1300 tracks the movement of the user 1350 and update the displayed content on the surround screen 1310 based on the movement of the user 1350. The display area may be 480″ wide by 67.5″ high, or 240″ wide by 67.5″ high, and the entire dimension may be 275″×138″×99″ or 243″×100″×32.5″ depending on the configuration. The displays 1311, 1312, and 1313 may be cart-mounted to form a wall in a free space.

The immersive system 1300 may be utilized as a visualization tool or an educational tool. A virtualized interactive medical, scientific, and entertainment content can be delivered to the user 1350. While the surround screen 1310 is shown in a three-wall configuration covering left, front, and right side of a room in the present example, the dimensions, resolution, configuration, the number of sides, shape and sizes of individual flat portions of the surround screen are not limited thereto, and any dimensions, configurations, number of sides, shape and sizes of individual flat portions of the large screen may vary without deviating from the scope of the present disclosure.

According to one embodiment, the surround screen 1310 may show a virtual figure (e.g., a patient, a nurse, an instructor) in one of the displays. The immersive system 1300 provides speech recognition to allow the user 1350 to interact with the virtual figure. The virtual figure may be AI-generated. Although the example of FIG. 13 shows only one user 1350, more users may sit, stand, or move around in an area at or vicinity of the center of the large screen to experience the content displayed thereon.

According to one embodiment, the contents displayed on the surround screen 1310 may be controlled with one or more control devices. In one embodiment, the immersive system 1300 may include various sensors and control devices such as a touch sensor, a motion sensor, a remote control, a joystick, a ring, a virtual reality (VR) device, a microphone, a keyboard, a voice recognition device, a camera, a touchscreen, a trackpad, mouse, a game controller, a foot pedal, or any combination thereof. The content displayed on or application running on the surround screen 1310 may be controlled with one or more of these input devices independently or in combination with the touch sensor or the motion sensor of the immersive system 1300. In another embodiment, a motion tracking device may be used to detect a gesture, motion, and/or signal of the user 1350 independently or in conjunction with other control devices. The user 1360 can navigate a digital scene displayed on the surround screen 1310 for controlling a viewpoint, selecting a question, and explore the scene interactively with the displayed objects.

FIG. 14 is a block diagram of an exemplary computing system suitable for implementing an embodiment of the present system. The computing system 1400 provides various contents and features of the digital table 200 of FIG. 2 and the immersive system 1300 of FIG. 13 as disclosed herein. The computing system 1400 may be representative of a computing device, such as a personal computer, a server, a mobile device, or a specialized hardware platform configured to perform the methods described herein.

In one embodiment, the computer system 1400 may include a processor 1410, a memory 1420, a storage device 1430, a communication interface 1440, and one or more input/output (I/O) devices 1450.

The processor 1410 may include a central processing unit (CPU), a graphics processing unit (GPU), a microcontroller, or any combination thereof. The processor 1410 may be any commercially available processor and is configured to execute computer-executable instructions and connected to a system bus that interconnects various components of the computing system and carries data and control signals. The memory 1420 may include volatile memory such as random-access memory (RAM), as well as non-volatile memory.

The storage device 1430 may include a hard disk drive (HDD), a solid-state drive (SSD), an optical disk, or other computer-readable media. An operating system may be typically stored on the storage device 1430 to manage the hardware and provide a platform for the application programs. The communication interface 1440 may enable wired or wireless data transfer with external systems via protocols such as Ethernet, Wi-Fi, or Bluetooth. The communication interface 1440 may also connect the computing system 1400 to one or more networks, such as a Local Area Network (LAN), a Wide Area Network (WAN), or the Internet. The I/O devices 1450 may include a display, a monitor, a microphone, a keyboard, a mouse, a touchscreen, a speaker, a printer, or other peripheral devices.

In operation, instructions for software programs may be stored in the memory 1420 or the storage device 1430 and executed by the processor 1410 to perform one or more methods as described herein. It is understood by a person having ordinary skill in the art that the computing system 1400 is for illustrative purposes only, and the present disclosure is not limited to this specific configuration. The inventive concept of the present disclosure may be implemented on any suitable computer system.

The computing system 1400 may correspond to a computer of the immersive system 1300 or the digital table 200 or may be used in conjunction with other computing hardware to run a wide variety of software and application programs. For example, the immersive system 1300 displays interactive environments on the surround screen 1310, and the user 1350 can explore a biological ecosystem, a space, and underwater world, conduct a science experiment, and perform a science quest. Examples of such interactive environments include, but not limited to, Plant Biome, Underwater Biome, City Explorer, Weather Satellite, Body Journey, etc. The immersive system 1300 or the digital table 200 may also be configured to run a science experiment of STEM topics across biology, chemistry, physics, and ESS.

According to one embodiment, the immersive system 1300 may be configured to explore a digital anatomy. For example, a real human anatomy exploration software is available from Anatomage Table™, that has a variety of human physiology collections, pathological radiology collections, clinical procedures, medical scenarios, etc.

A body journey application allows the user 1350 to navigate through a digital human (or animal) body by providing an immersive experience and interaction with various anatomical features displayed on the surround screen 1310. The scale of anatomical features displayed on the surround screen 1310 may be adjusted or set to allow the user 1350 to navigate through various anatomical features of the human body such as vascular, arteries and vein structures, organs, bones, muscles, nerves, etc. In this manner, the user 1350 can perceive the journey as being transformed into a cell, a microrobot, or a virtual being that is capable of imaginarily passing through the anatomical features of the digital body. The user 1350 perceives the visualization of the anatomical features so immersively and engaging in a manner that has never seen before or experienced. As opposed to learning from demonstrated images from the textbook, the user 1350 can better understand the anatomical structures, connections, and passages as well as physiological principles from a perspective of a non-human being at a microscopic scale that would be difficult or impossible to perceive or even imagine at a real scale.

The human anatomy displayed on the surround screen 1310 in the body journey application may represent a real human anatomy. For example, the real human anatomy may be digitally scanned from a human cadaver or reconstructed from medical images such as CT scans, MRI, etc. The human anatomy can demonstrate physiological functions of living bodies as realistically as possible compared to a computer-generated anatomy model that lacks reality. According to one embodiment, the real human anatomy may be available by Anatomage Table™ or Anatomage eBook of Anatomage Inc, San Jose, California.

According to one embodiment, the body journey application asks the user 1350 to perform a diagnosis or take a therapeutic action. The symptom and therapeutic procedures may be clinically meaningful realistic particularly when the anatomy displayed on the surround screen 1310 is real human anatomy.

According to one embodiment, the immersive system 1300 may be configured to run various exploration applications. For example, in the body journey application, the user 1350 is given a task (or a mission) to find a shortest or fastest passage from one location (e.g., inside a vein in a foot) to another location (e.g., the left ventricle) within the digital human body. This may require prior knowledge and understanding of the human anatomy, and it may be used as a quiz or test in a medical or nursing school.

In another example, the user 1350 may be put into a situation of emergency that requires an immediate medical action or treatment. In this case, the task given to the user 1350 may need to be completed within allowable time limit. A virtual patient may exhibit a symptom or have a particular medical condition (e.g., a severe chest pain, a patient arrived from a car accident, a patient having imminent cerebral hemorrhage, a patient experiencing myocardial infarction). The user 1350 may commence the task when ready. The user 1350 may be given a visual cue (e.g., a blinking red emergency sign with a timer counting down) to promptly assess and diagnose the virtual patient's condition. The user 1350 can switch the view from an emergency room to a view of the patient's particular body part via a voice command. Alternatively, the user 1350 may be directed to a random body location of the patient, and the task is to navigate from the current location (e.g., within a vein in the left leg) to a location where an appropriate action needs to be taken (e.g., removing a clot) based on the patient's condition. The task may be a practical and clinically meaningful or critical one, and the user 1350 may require taking a series of actions and making time-sensitive decisions as a part of medical education and training.

For example, the virtual patient has cerebral hemorrhage. In this case, the user 1350 may need to “virtually” remove a section of the patient's skull by acting on the virtual patient using one or more virtual medical devices or instruments. This may be conceived as playing a surgeon's role in a medical video game. The user 1350 may use those tools and voice commands to access various body parts and organs of the virtual patient, and take actions (e.g., removing a clot). These virtual and interactive experiences may not be obtained by merely learning from a textbook or dissecting a cadaver. In that regard, the immersive system 1300 implementing this training methodology has tremendous educational benefits as the user 1350 would gain deeper medical knowledge of the human anatomy as well as develop an ability as a medical professional to properly diagnose a condition of a patient and promptly treat the patient. The immersive system 1300 may implement various tasks and tests to train the user 1350 while providing realistic and interactive user experiences to the real human anatomy in a more engaging and exciting manner.

In another embodiment, the immersive system 1300 may be configured to explore a virtual hospital. The virtual hospital may include rooms and facilities such as a reception area, an administration department, a billing department, a conference room, a training room, cafeteria, an emergency room, a patient ward, an intensive care unit, an operating room, pharmacy, an imaging room, etc.

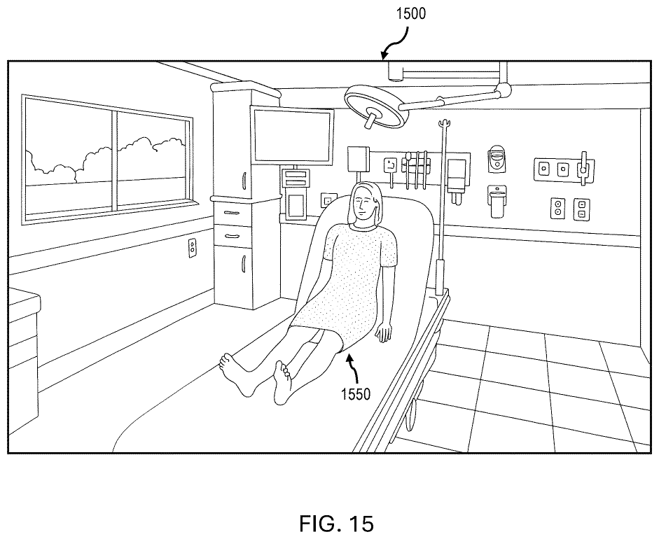

FIG. 15 illustrates an example of a virtual hospital room, according to one embodiment. The immersive system 1300 of FIG. 13 may display a scene 1500 of the virtual hospital room on the surround screen 1310. The scene 1500 offers an immersive, interactive, and photo-realistic hospital environment for a trainee. The scene 1500 may include a virtual patient 1550.

The virtual patient 1550 is a computer-based AI simulation of a human patient that can converse interactively with a medical trainee, mimicking real clinical encounters. Instead of scripted or one-way case studies, the immersive system 1300 adopts natural language processing (NLP) and speech recognition technology so the trainee can ask questions, take histories, or discuss diagnoses just as they would with a real patient.

Although it is not explicitly shown, other people such as a medical assistant or a nurse may also appear in the scene 1500. The user 1350 can verbally interact and converse with them to ask questions, review and discuss the patient's charts and test results, order a test, administer medications, and even interact with an equipment that is available in the virtual hospital room. For example, the user 1350 may ask for a medical chart and/or images (e.g., CT, MRI, X-ray) of the virtual patient, and a nurse prepares and put them up to one of the displays of the surround screen 1310. Different views and displays may be shown to each of the three displays 1311, 1312, and 1313 of the surround screen 1310. For example, the display 1311 may show medical images of the virtual patient, the display 1312 may show the virtual hospital room in which the virtual patient 1550 is, and the display 1313 may show vital signs of the virtual patient 1550.

The immersive reality of the virtual hospital room may be further augmented by one or more tangible physical medical tools and equipment (e.g., stethoscope, sphygmomanometer, pulse oximeter, otoscope, oxygen mask) so that the actual trainee who is represented by the user 1350 can physically interact with them. A counterpart digital model of the tool or equipment may appear in the displays of the surround screen 1310, and its motion and orientation may be synchronized with the physical tool that the actual trainee touches. This way, the trainee can experience a more realistic and interactive hospital environment by way of combining true reality into the virtually real hospital model.

The virtual patient 1550 may be generated from an AI model based on actual clinical data to better represent an actual patient that the trainee may encounter. For example, a virtual patient model may be generated using Convai plugin from Unreal Engine. Convai is a system that integrates advanced, conversational AI, allowing the virtual patient 1550 to have open-ended, intelligent conversations, respond to real-world events and the user's commands, and animate a speech with lip-sync and facial expressions.

The virtual hospital including the virtual patient may also be computer-generated with a real clinical data. After initial generation, the virtual hospital model may be reviewed, refined, continuously updated, and stored in a database (DB). Many virtual patients with many different symptoms and conditions can be included in the DB, and the trainee may be trained with them. In a training session, a particular virtual hospital model may be loaded from the DB based on a user's selection. The virtual hospital model provides data-centric, interactive, and hyper realistic user experience so that trainee perceives seeing and interacting with a real patient in real-life situation.

The virtual hospital model may be constructed and stored in a DB schema template format. The model virtual hospital may incorporate various clinical data including case details, patient, exam, stabilize, differential, investigate, intervene, communicate, and hand-off, etc. The case details DB may include a case ID, a case release date, a description, continuing medical education (CME) eligibility, categories, a chief complaint, an author, a peer review, a recent score, and a best score, etc. The patient DB may include a name, an age, height, weight, code status, EMS report, a triage nurse, a chief complaint, conversations, etc. The exam DB may include various status and results of various examinations such as airway, breath, circulation, neck, cardiovascular, pulmonary, abdominal, gastrointestinal, and respective evaluations and user actions for each exam. The stabilize DB may include various treatments performed on the virtual patient to stabilize symptoms and medical conditions. The differential DB may include medical conditions of the virtual patient under various categories such as allergy/immune, cardiovascular, dermatological, endocrine/metabolic, environmental, gastrointestinal (GI), hematology/oncology, infectious disease, musculoskeletal/orthopedic, neurological, obstetrical/gynecological (OB/GYN), ophthalmological, otolaryngological (ear, nose and throat), pulmonary/thoracic, renal/genitourinary, toxicological, trauma/vascular, etc. The investigate DB may include lab results such as blood sugar level, cell counts, CT and MRI imaging, etc. The intervene DB may include medical treatments and administered medications, etc. and the respective evaluations and user actions. The communicate DB may include consultation with specialists such as cardiology, endocrinology, gastroenterology, surgeon, oncologist, neurologist, neurosurgeon, psychiatrist, pulmonologist, etc. The hand-off DB may include admittance, follow-ups, and discharge from the operating room and/or the virtual hospital etc.

In another embodiment, the immersive system 1300 may be configured to provide exploration experiences. An example of such exploration experiences is an immersive space exploration application. The user 1350 may virtually fly in a spacecraft and discover and interact with various astronomical objects such as planets, stars, asteroids, comets, moons, black holes, and galaxies. The astronomical objects may be constructed with real data that may be obtained from the NASA's database or from a fictitious model.

In the space exploration application, the user 1350 may be give a mission to explore a new planet where geological and atmospheric properties and environments are so much different from the Earth. The new planet may present extreme surviving challenges, and the user 1350 may be given visual and/or auditory cues to properly assess the surrounding conditions, properties, and characteristics of the new plant. Alike a video gaming environment, the user 1350 may need to take proper actions to overcome the extreme challenges and survive and settle on the new planet. The immersive system 1300 may provide various challenges and goals to invoke the user's prompt responses and actions and test their knowledge. During or at the completion of the mission, the user 1350 may be assessed with a score based on his/her individual performance or a group's collaborative performance. The score may be awarded based on the time to resolve the challenges, actions taken, and/or decisions made. The immersive system 1300 may be configured to provide various other exploration experiences, for example, a spacecraft launching, orbiting a planet or a moon, and landing back to Earth.

In another embodiment, the immersive system 1300 may be configured to explore the Earth from a weather satellite orbiting the Earth. From the satellite, the user 1350 can observe various weather conditions of the Earth (e.g., clouds, pressure, wind, temperature, rain) as well as natural disasters or anomaly such as hurricane, typhoon, cyclone, wildfire, volcano, and oil spills. The user 1350 may be able to virtually switch to another satellite and move along the orbit of the Earth changing their viewpoint. The switching of the viewpoint between different satellites may occur using a cinematic camera transition technique, for example, in a slow motion with zooming in and/or out. Examples of the missions given to the user 1350 may include, but are not limited to, identifying an event or incident that may occur randomly during the mission, logging incidents, navigating the satellite, observing changes over time, reporting and alerting to a dispatch responder.

The data for various weather conditions that are observable by the user 1350 may be generated from real satellite data. Examples of such data include, but are not limited to, cloud and atmospheric moisture data, temperature, wind, precipitation, sea surface data, pressure data, lightening data, aerosols and particulates data, solar radiation and energy flux, real-time storm monitoring data.

To control orientation and attitude of the satellite, the user 1350 can use reaction wheels that spin at high speeds to create angular momentum. The reaction wheels can change the satellite's orientation by electric power without consuming fuel. The user 1350 can use the reaction wheels for precise, continuous adjustments to align sensors and instruments with specific targets or maintain the satellite's stability. A magnetorquer use electromagnetic coils to interact with Earth's magnetic field, generating torque to adjust the satellite's orientation. The user 1350 can use the magnetorquer equipped with the satellite to dump excess angular momentum from the reaction wheels or make coarse adjustments. The magnetorquer consume very little energy and do not rely on fuel. The user 1350 can also use a thruster to adjust the satellite's position or orientation. The truster fires a burst of gas or monopropellant (like hydrazine) and it is typically used for larger maneuvers, such as orbit corrections, emergency adjustments, or countering disturbances that the reaction wheels and the magnetorquer cannot handle. The thruster should be used sparingly to conserve the onboard fuel supply.

In another embodiment, the immersive system 1300 may be configured to explore a virtual city. The user 1350 can explore a digitally modeled virtual city (e.g., Rome, Paris, New York) by interacting with various objects and features presented therein such as buildings, structures, bridges, rivers, parks, etc. In one embodiment, a scientific task associated with a building in the virtual city may be presented to the user 1350. The task given to the user 1350 may involve performing a scientific experiment, simulation, and/or answering a question or solving a quiz. As the user 1350 successfully resolves the given task or mission, the user 1350 may be awarded with more resources or the infrastructure of the virtual city may improve. For example, a visual improvement to the virtual city may emotionally awarding to the user 1350 as the mission successfully continues. The user or a group of users may continue to improve the city by engaging with more educational and/or scientific activities such as performing scientific experiments while navigating through the virtual city and overcoming various challenges and environmental disasters such as global climate change, floods, storms, droughts, etc.

In another embodiment, the immersive system 1300 may provide a biological ecosystem application. The user 1350 may experience and interact with a visually realistic environment as well as various plants, insects, and animals in various ecosystems or climatic. The user 1350 can explore various biological ecosystem environment whether that is realistic (e.g., Amazon rainforest) or manufactured (e.g., an alien planet; see the example described above regarding the space exploration application). The user 1350 may be given a task or mission to discover specific plants, insects and/or animals, collect samples, and grow the samples to run a scientific experiment. This scientific experiment may be performed virtually with visual and/or auditory cues.

The biological ecosystem application allows the user 1350 to explore, identify, collect, and investigate plants. Herein, the biological ecosystem application may also be referred to as a plant-focused biological world application or a plant biome application. The weather conditions and changes of terrains and environmental factors such as a rain effect, a time of the day, and a seasonal effect can provide dynamic yet realistic immersive experience to the user 1350.

According to one embodiment, the scenes of the biological ecosystem application may be generated using Unreal Engine's Procedural Content Generation (PCG) plugin and nDisplay that are commercially available. The image quality and performance of the scenes may be further augmented by NVidia's DLSS that is an AI-powered technology to improves anti-aliasing, ray tracing, lighting, frame rate, etc.

According to one embodiment, the plant biome application may be gamified as a multiplayer game. A team selection menu is provided to create or select a team. The team selection menu also loads an existing team's profile and their past playing history or allows a new team to create their team profile. After the team profile is selected, created, or loaded, the players connect their controllers to the computer with and join the game as a captain (or player 1), player 2, player 3, and player 4, and so on. The players can change their settings such as name, cursor color, and design from a player setting menu.

Each player can take on a specific role in the game, for example, a soil specialist and a data analyst. The team can assign specialties to the players to ensure that everyone contributes uniquely to the team. Alternatively, an instructor (e.g., a teacher) can assign a specific role to a player. After every player confirms their selection and is ready, the game start by pressing a start button.

The team may begin their journey by entering a specific scene, (e.g., meadow, tropical rainforest, desert, savannah, shrubland, Taiga, temperate rainforest, temperate grassland, tundra), If the team is logged in, the previous scene may load to resume playing the game. The players may be equipped with various tools such as a soil moisture meter, a thermometer, and a light meter.

According to one embodiment, the team's journey may be associated with a task or a mission. In general, the team may be tasked to identify and collect plant specimens while documenting the environment using a data collection tool. However, plant information such as temperature, sunlight, moisture level, etc. may not populate in the team's collection or inventory until the team records data for themselves with their own equipment. When the player hovers a crosshair over a plant, if they discovered before, its name/image appears to inform that the play has been already collected, or a message that a new plant has been found.

Back at their base camp, the team may analyze the plants that they collected and hypothesize about their ideal growth conditions. Based on the analysis of the data and their knowledge, the team may formulate a hypothesis for each collected plant's ideal growth conditions.

According to one embodiment, the plant biome application may provide a tool referred to as Hypothesis Builder. This in-game tool guides the players to execute a task or mission. The task or mission may be given as a hint such as “This plant thrives in high fertility, moderate soil wetness, and temperatures between 22° C. and 25° C.” Based on the given task or mission, the players scatter, collect plants, bring their seeds back to the base camp, and they test their hypotheses in a controlled greenhouse environment.

To perform the test, the team may be provided with an experiment design tool. Using the experiment design tool, the team can set up a growth chamber to simulate their hypothesized conditions (e.g., adjustable light levels, soil types, and irrigation). The growth of the plant may be observed regularly (e.g., daily, weekly, monthly) with notifications (e.g., “leaves are wilting due to low soil wetness” or “flowering has started due to optimal conditions.”). The players may utilize other visual tools to create a heatmap or a graph showing the soil wetness, fertility, and temperature variation across the biome.

Each plant's growth may be graded based on a growth rate (e.g., how quickly the plant reaches maturity), the plant's health (e.g., leaf color, flowering, and seed production, and reproducibility), and success in gathering new seeds. The players (or the team) can earn scores, and they can use them to upgrade their equipment or acquire biome-specific tools (e.g., an advanced moisture sensor for desert or an UV meter useful in a tropical region).

For new a new player, the plant biome application may start with a tutorial session. The player can get familiarized with controls and interfaces and practice with actions such as how to collect. This may be helpful for a first-time player to learn how the game works. The tutorial may appear by default for a newly created team. The initial load for a new team may show an available in-game asset (e.g., greenhouse, tools) or inform the team players.

After entering a scene of the game, the player is reminded of the game objectives on the screen. The game may have well predefined goals or objectives for the player to work towards. The fun part of the game starts with well-defined goals/objectives to tightly engage the player instead of merely wandering and collecting plants with no specific goals/objectives.

The plant biome application may provide a reward system that rewards the players for playing the game. The higher the player's level, the more accomplished the player feels when progressing through the game. The plant biome application may provide a leaderboard that can be viewed by the players to encourage them to eagerly find more plants to rise in the ranks in the leaderboard. The plant biome application may have a timed task (e.g., daily, weekly) for the players. The players are given a task to find a plant within the time limit.

When a player successfully takes a picture of a plant, a popup window may appear to inform the player that the plant has been collected with a message saying, “You found plant name!” Then, the player presses a resume button to resume the game.

While searching through the biological ecosystem, the user 1350 may be given a task to discover a specific plant. Based on a grade, level, experience, or depth of knowledge of the user 1350, an appropriate task of challenges, missions, and goals may be provided to prompt continuous interests and joyfully engage with the given task. Successful completion of the task would be rewarded, for example, upgrading levels or awarding points. This way, the user 1350 may progressively learn while actively and interactively engaging with the topic and obtaining knowledge.

From an educator's perspective, the plant-focused biological world application may provide a new educational paradigm potentially replacing conventional biology education. The objectives of the plant-focused biological world application include, but not limited to, learn the types of plants, learn a biological mechanism of the plants, learning reproductive and pollination of the plants, and learn the environment associated with the plants.

The user 1350 (e.g., student) may be tasked to find a specific plant, find a plant that has certain biological characteristics, or find a plant based on a hint or a prompting question while navigating the ecosystem. For example, after stung by an insect or touching a poisonous plant, the user 1350 is tasked to find another plant that can neutralize the poison or mitigate the pain. Other types of tasks and/or challenges may be posed to the user 1350 while navigating through the ecosystem, and the user's activity may be evaluated or assessed based on his/her responses and actions.

According to one embodiment, more than one user (e.g., student, trainee) may collaborate to solve quizzes, missions, or challenges presented by the immersive system 1300. The evaluation may be given based on collective scores of the 5s for a given set of quizzes or challenges. Several user groups may compete in a competition, and the winner may be determined based on the collective scores. In one embodiment, a subgroup of a group of users may be given one task while other subgroup may be given another task, and the group may be awarded with a score based on their problem-solving skills and tactical approaches and how their collaborative goal has reached.

According to one embodiment, the immersive system 1300 may provide a virtual scene of an ecosystem that is specifically designed for a target application. The ecosystem may include diverse plants that are embedded biologically accurately for specific climate conditions. The plants may include a variety of plants that are not only indigenous species and plants as well as alien species and plants that can introduce some environmental challenges. These variety of plants can provide more diverse and enriched ecological settings.

According to one embodiment, the immersive system 1300 may place various plants based on the proper environmental conditions of the target ecosystem, for example, humidity, sunlight, wind, altitude, geological weather patterns, seasons, time of a day, etc. The user 1350 may have encounter virtual interactions with the plants by touching, pushing, tearing, and picking, and can run an experiment with them to learn more about the plants.

According to one embodiment, the immersive system 1300 may provide weather simulations such as rain, fog, water, humidity change, wind to the target ecosystem. The immersive system 1300 may also provide natural simulations including non-interacting background species and animals such as birds, bees, and bugs, river, sunlight, sound, etc. Examples of the interactive plants included in the plant-focused biological world application may include, but are not limited to, algae, mosses, ferns, conifers, fungi, flower plants, fruit trees, grasses, bromeliaceae, orchids, monocots, eudicots, cacti, Brassica oleracea.

According to one embodiment, the immersive system 1300 may provide exciting and engaging activities for the user 1350. The plants may include common edible vegetable and fruits in their natural forms that may look different from those that the user 1350 may be familiar with. More exotic and visually attractive plants may be displayed to trigger more interests for the 1350 to explore them.

According to one embodiment, the immersive system 1300 may provide a blooming animation of a plant. When interacting with the plant, a time scale associated with the blooming animation may be adjusted in conjunction with the surrounding ecosystem to provide time-accelerated user experience.

According to one embodiment, the immersive system 1300 may be configured to explore underwater. Ther underwater application allows the user 1350 to experience environment under the sea. To provide the educational benefit through entertainment, a group of players are given a mission to explore a deep sea. The group may include a captain, a navigator, a collector, a data logger, etc. The captain is responsible for the mission and work with others to make mission-critical decisions during the journey. The navigator navigates a submarine to a destination (e.g., a trench) using a sonar. The collector controls robotic arms to collect various objects during the mission. The data logger operates a camera and records videos and pictures of sea animals and plants. The data logger may also monitor the pressure, temperature, oxygen level, pH level, etc. of the sea.

As the submarine navigates to its destination (e.g., the deep trench), they may find sea animals and plants and perform scientific investigations on them. Finding a rare one may be challenging due to the harsh environment. During the mission, the user 1350 can learn how to interpret interactions between geological and ecological systems and how hydrothermal vents and other geological features affect the surrounding marine ecosystems. At the end of the mission, the user 1350 may develop a model may that describes the flow of matter and energy in the trench ecosystem, focusing on food webs and nutrient cycling.

The user 1350 can also assess extreme environmental factors such as high pressure and low temperature in the trench can influence animal and plant population dynamics and resilience of the ecosystem. The user 1350 may learn how adaptations such as bioluminescence and pressure resistance enable organisms to survive in such extreme conditions. Further, the user 1350 can evaluate how past geologic processes influenced the uneven distribution of geological resources in the trench by analyzing the geological features and processes of the deep trench, including plate tectonics, subduction zones, and seafloor spreading that can explain Earth's dynamic crust formation. The user 1350 can also synthesize evidence of plate movements to understand the formation and evolution of ocean floor features.

According to one embodiment, an immersive system includes: a computing system including a microphone and a speaker; and a surround screen. The computing system provides interactive scenes of a virtual hospital including a virtual patient on the surround screen. The virtual hospital includes one or more of hospital facilities, and a user is adapted to move around the one or more of hospital facilities. The virtual patient has a symptom or has a medical condition, and the user is further adapted to interact with the virtual patient via a voice command input through the microphone, an auditory output from the speaker, and the interactive scenes displayed by the surround screen.

The virtual patient may be an Artificial Intelligence (AI)-generated patient based on clinical patient data.

The surround screen may display a medical chart or a medical image of the virtual patient at a request of the user.

The immersive system may further include a medical tool that the user can touch and manipulate.

A digital counterpart of the medical tool may be updated on the interactive scenes displayed on the surround screen based on a position and an orientation of the medical tool.

The immersive system may further include a plurality of input devices to receive an input from the user, wherein the plurality of input devices includes one or more of a remote control, a joystick, a ring, a virtual reality (VR) device, a keyboard, a camera, a touchscreen, a trackpad, mouse, a game controller, and a foot pedal.

Based on consultation with the virtual patient, the user may treat the virtual patient with a medication, order a test, or a medical procedure.

The user's treatment on the virtual patient may be graded in a medical training session.

The one or more of hospital facilities may include a reception area, an administration department, a billing department, a conference room, a training room, cafeteria, an emergency room, a patient ward, an intensive care unit, an operating room, pharmacy, or an imaging room.

The surround screen may include a first display positioned on a left side, a second display positioned on a right side, and a third display positioned between the first display and the second display, and the first display, the second display, and the third display provide 270-degree immersive experience to the user.

According to one embodiment, a method of providing an immersive experience to a user includes: providing a computing system that includes a microphone and a speaker and a surround screen; providing interactive scenes of a virtual hospital including a virtual patient on the surround screen, wherein the virtual patient has a symptom or has a medical condition; and updating the scenes of the virtual hospital based on interaction between the user and the virtual patient via a voice command input through the microphone, an auditory output from the speaker, and the interactive scenes displayed by the surround screen. The virtual hospital includes one or more of hospital facilities, and a user is adapted to move around the one or more of hospital facilities.

The virtual patient may be an Artificial Intelligence (AI)-generated patient based on clinical patient data.

The method may further include displaying a medical chart or a medical image of the virtual patient on the surround screen at a request of the user.

The method may further include receiving an input from the user using a medical tool that can be touched and manipulated by the user.

The method may further include displaying and updating a digital counterpart of the medical tool on the interactive scenes on the surround screen based on a position and an orientation of the medical tool.

The method may further include receiving an input from the user using a plurality of input devices, wherein the plurality of input devices includes one or more of a remote control, a joystick, a ring, a virtual reality (VR) device, a keyboard, a camera, a touchscreen, a trackpad, mouse, a game controller, and a foot pedal.

The method may further include receiving an input from the user after consulting with the virtual patient, the input relates to treating the virtual patient with a medication, order a test, or a medical procedure.

The method may further include grading the user's treatment on the virtual patient in a medical training session.

The one or more of hospital facilities may include a reception area, an administration department, a billing department, a conference room, a training room, cafeteria, an emergency room, a patient ward, an intensive care unit, an operating room, pharmacy, or an imaging room.

The surround screen may include a first display positioned on a left side, a second display positioned on a right side, and a third display positioned between the first display and the second display, and the first display, the second display, and the third display provide 270-degree immersive experience to the user.

A system and method for providing interactive educational content and immersive learning and training experience are disclosed. Although various embodiments have been described with respect to specific examples and subsystems, it will be apparent to those of ordinary skill in the art that the inventive concepts disclosed herein are not limited to these specific examples or subsystems but extends to other embodiments as well. Included within the scope of these concepts are all of these other embodiments as specified in the claims that follow.

What is claimed is:

1. An immersive system comprising:
a computing system including a microphone and a speaker; and
a surround screen,
wherein the computing system provides interactive scenes of a virtual hospital including a virtual patient on the surround screen,
wherein the virtual hospital includes one or more of hospital facilities, and a user is adapted to move around the one or more of hospital facilities, and
wherein the virtual patient has a symptom or has a medical condition, and the user is further adapted to interact with the virtual patient via a voice command input through the microphone, an auditory output from the speaker, and the interactive scenes displayed by the surround screen.

2. The immersive system of claim 1, wherein the virtual patient is an Artificial Intelligence (AI)-generated patient based on clinical patient data.

3. The immersive system of claim 1, wherein the surround screen displays a medical chart or a medical image of the virtual patient at a request of the user.

4. The immersive system of claim 1 further comprises a medical tool that the user can touch and manipulate.

5. The immersive system of claim 4, wherein a digital counterpart of the medical tool is updated on the interactive scenes displayed on the surround screen based on a position and an orientation of the medical tool.

6. The immersive system of claim 1 further comprises a plurality of input devices to receive an input from the user, wherein the plurality of input devices includes one or more of a remote control, a joystick, a ring, a virtual reality (VR) device, a keyboard, a camera, a touchscreen, a trackpad, mouse, a game controller, and a foot pedal.

7. The immersive system of claim 1, wherein based on consultation with the virtual patient, the user treats the virtual patient with a medication, order a test, or a medical procedure.

8. The immersive system of claim 7, wherein the user's treatment on the virtual patient is graded in a medical training session.

9. The immersive system of claim 1, wherein the one or more of hospital facilities include a reception area, an administration department, a billing department, a conference room, a training room, cafeteria, an emergency room, a patient ward, an intensive care unit, an operating room, pharmacy, or an imaging room.

10. The immersive system of claim 1, wherein the surround screen includes a first display positioned on a left side, a second display positioned on a right side, and a third display positioned between the first display and the second display, and the first display, the second display, and the third display provide 270-degree immersive experience to the user.

11. A method of providing an immersive experience to a user, the method comprising:
providing a computing system that includes a microphone and a speaker and a surround screen;
providing interactive scenes of a virtual hospital including a virtual patient on the surround screen, wherein the virtual patient has a symptom or has a medical condition; and
updating the scenes of the virtual hospital based on interaction between the user and the virtual patient via a voice command input through the microphone, an auditory output from the speaker, and the interactive scenes displayed by the surround screen,
wherein the virtual hospital includes one or more of hospital facilities, and a user is adapted to move around the one or more of hospital facilities.

12. The method of claim 11, wherein the virtual patient is an Artificial Intelligence (AI)-generated patient based on clinical patient data.

13. The method of claim 11 further comprises: displaying a medical chart or a medical image of the virtual patient on the surround screen at a request of the user.

14. The method of claim 11 further comprises: receiving an input from the user using a medical tool that can be touched and manipulated by the user.

15. The method of claim 14 further comprises: displaying and updating a digital counterpart of the medical tool on the interactive scenes on the surround screen based on a position and an orientation of the medical tool.

16. The method of claim 11 further comprises receiving an input from the user using a plurality of input devices, wherein the plurality of input devices includes one or more of a remote control, a joystick, a ring, a virtual reality (VR) device, a keyboard, a camera, a touchscreen, a trackpad, mouse, a game controller, and a foot pedal.

17. The method of claim 11 further comprising receiving an input from the user after consulting with the virtual patient, the input relates to treating the virtual patient with a medication, order a test, or a medical procedure.

18. The method of claim 17 further comprising grading the user's treatment on the virtual patient in a medical training session.

19. The method of claim 11, wherein the one or more of hospital facilities include a reception area, an administration department, a billing department, a conference room, a training room, cafeteria, an emergency room, a patient ward, an intensive care unit, an operating room, pharmacy, or an imaging room.

20. The method of claim 11, wherein the surround screen includes a first display positioned on a left side, a second display positioned on a right side, and a third display positioned between the first display and the second display, and the first display, the second display, and the third display provide 270-degree immersive experience to the user.
